(19) United States
(12) Patent Application Publication
(10) Pub. No.: US 2022/0220543 A1
(43) Pub. Date: Jul. 14, 2022

(54) METHODS AND REAGENTS FOR NUCLEIC ACID SEQUENCING AND ASSOCIATED APPLICATIONS

(71) Applicant: TwinStrand Biosciences, Inc., Seattle, WA (US)

(72) Inventor: Jesse J. SALK, Seattle, WA (US)

(21) Appl. No.: 17/607,490

(22) PCT Filed: Aug. 1, 2020
(86) PCT No.: PCT/US2020/044673

§ 371 Date: Oct. 29, 2021

Related U.S. Application Data
(60) Provisional application No. 62/881,936, filed on Aug. 1, 2019.

(51) Int. Cl. C12Q 1/6827; C12Q 1/6869

(52) U.S. Cl. CPC C12Q 1/6827; C12Q 1/6869

(57) ABSTRACT

The present technology relates generally to the methods and associated reagents for providing error-corrected nucleic acid sequences. In particular, several embodiments are directed to adapter molecules comprising a hairpin shape and methods of use of such adapters in Duplex Sequencing and other sequencing applications. In some embodiments, physically-linked nucleic acid complexes comprising both the first strand and the second strand can be amplified and independently sequenced in a same clonal cluster on a sequencing surface.

### CROSS-REFERENCE TO RELATED APPLICATIONS

This application claims priority to and the benefit of U.S. Provisional Patent Application No. 62/881,936, filed Aug. 1, 2019, the disclosure of which is hereby incorporated by reference in its entirety.

### TECHNICAL FIELD

The present technology relates generally to the methods and associated reagents for providing high accuracy (e.g., error-corrected) nucleic acid sequences. In particular, several embodiments are directed to adapter molecules comprising a hairpin shape and methods of use of such adapters in Duplex Sequencing and other sequencing applications.

### BACKGROUND

Duplex Sequencing is an error-correction method that achieves exceptional sequence accuracy by comparing the sequence information derived from both strands of individual double-stranded nucleic acid molecules. With regard to the efficiency of a Duplex Sequencing process or other high-accuracy sequencing modalities, conversion efficiency can be defined as the fraction of unique nucleic acid molecules inputted into a sequencing library preparation reaction from which at least one duplex consensus sequence read (or other high-accuracy sequence read) is produced. In some instances, conversion efficiency shortcomings may limit the utility of high-accuracy sequencing for some applications where it would otherwise be very well suited. For example, a low conversion efficiency would result in a situation where the number of copies of a target double-stranded nucleic acid is limited, which may result in a less than desired amount of sequence information produced. There is a need for cost- and manufacture efficient methods in which to synthesize raw sequence reads of nucleic acid molecules for use in various applications, including for Duplex Sequencing applications.

### SUMMARY

The present technology relates generally to methods and associated reagents for nucleic acid sequencing. In particular, some aspects of the technology are directed to methods for achieving high accuracy sequencing reads that is provided at a faster rate (e.g., with fewer steps) and/or with less cost (e.g., utilizing fewer reagents), and resulting in increased desirable data. Other aspects of the technology are directed to methods and reagents for increasing conversion efficiency for Duplex Sequencing. Various aspects of the present technology have many applications in both pre-clinical and clinical testing and diagnostics as well as other applications.

In some aspects, the present disclosure provides methods of sequencing a double-stranded target nucleic acid molecule comprising the steps of: (a) amplifying a physically-linked nucleic acid complex on a surface to produce physically-linked nucleic acid complex amplicons bound to the surface in both a forward orientation and a reverse orientation, wherein the physically-linked nucleic acid complex comprises (i) the double-stranded target nucleic acid molecule, (ii) a first adapter comprising a linker domain on a first end of the double-stranded target nucleic acid molecule, and (iii) a second adapter having a double-stranded portion and a single-stranded portion on a second end of the double-stranded target nucleic acid molecule; (b) removing either (i) the physically-linked nucleic acid complex amplicons bound to the surface in the reverse orientation or (ii) the physically-linked nucleic acid complex amplicons bound to the surface in the forward orientation; (c) cleaving a portion of the remaining bound physically-linked nucleic acid complex amplicons to provide a subset of single-stranded amplicons comprising information from one strand and a subset of physically linked nucleic acid complex amplicons; (d) sequencing the subset of single-stranded amplicons to provide a sequencing read derived from an original strand of the double-stranded target nucleic acid molecule; (e) amplifying the subset of physically linked nucleic acid complex amplicons on the surface; (f) removing the physically-linked nucleic acid complex amplicons that are in the other orientation; (g) cleaving the remaining bound physically-linked nucleic acid complex amplicons to provide single-stranded amplicons comprising information from the other strand; and (h) sequencing the single-stranded amplicons to provide sequencing reads derived from the other original strand of the double-stranded target nucleic acid molecule.

In some aspects, the present disclosure provides methods of sequencing a double-stranded target nucleic acid molecule comprising the steps of: (a) amplifying a physically-linked nucleic acid complex on a surface to produce a cluster of physically-linked nucleic acid complex amplicons bound to the surface, wherein the physically-linked nucleic acid complex comprises (i) the double-stranded target nucleic acid molecule, (ii) a first adapter comprising a linker domain on one end of the double-stranded target nucleic acid molecule, and (iii) a second adapter having a double-stranded portion and a single-stranded portion on the other end of the double-stranded target nucleic acid molecule; (b) removing either the physically-linked nucleic acid complex amplicons bound to the surface at (i) a 5′ end of the physically-linked nucleic acid complex amplicons or (ii) a 3′ end of the physically-linked nucleic acid complex amplicons; (c) cleaving at least a portion of the remaining bound physically-linked nucleic acid complex amplicons at a cleavage site to provide single-stranded amplicons comprising sequence information derived from one original strand of the double-stranded target nucleic acid molecule; and (d) sequencing the single-stranded amplicons to provide a sequencing read derived from the one original strand of the double-stranded target nucleic acid molecule. In some aspects, the method further comprises cleaving at least a portion of the remaining bound physically-linked nucleic acid complex amplicons comprises preserving at least one physically-linked nucleic acid complex amplicon bound to the surface. In some aspects, the method further comprises the steps of (e) amplifying the at least one physically-linked nucleic acid complex amplicon on the surface to repopulate the cluster of physically-linked nucleic acid complex amplicons bound to the surface; (f) removing the physically-linked nucleic acid complex amplicons that are in the other orientation not removed in (b); (g) cleaving the remaining bound physically-linked nucleic acid complex amplicons to provide single-stranded amplicons comprising information derived from the other original strand of the double-stranded target nucleic acid molecule; and (h) sequencing the single-stranded amplicons to provide a sequencing read derived from the other original strand of the double-stranded target nucleic acid molecule.

In some aspects, the methods further comprise the step of comparing the sequence read from the one original strand to the sequence read from the other original strand to generate a consensus sequence for the double-stranded target nucleic acid molecule. In some aspects, the methods further comprise the steps of identifying sequence variations in the sequence read from the one original strand and the sequence read from the other original strand, wherein the sequence variations from the one original strand and the other original strand are consistent sequence variations; or eliminating or discounting sequence variations that occur in the one original strand and not the other original strand. In some aspects, the methods further comprise the steps of comparing the sequence read from the one original strand to the sequence read from the other original strand; identifying a nucleotide position that does not agree between the sequence read from the one original strand to the sequence read from the other original strand; and generating an error-corrected sequence of the double-stranded target nucleic acid molecule by discounting. eliminating, or correcting the nucleotide position identified that does not agree.

In some aspects, the present disclosure provides methods of sequencing a population of double-stranded target nucleic acid molecules, each comprising a first strand and a second strand, comprising the steps of: (a) amplifying a plurality of physically-linked nucleic acid complexes on a surface to produce a plurality of clonal clusters, each clonal cluster comprising a plurality of physically-linked nucleic acid complex amplicons each comprising a first strand amplicon and a second strand amplicon, wherein each physically-linked nucleic acid complex comprises (i) a double-stranded target nucleic acid molecule from the population, (ii) a first adapter comprising a linker domain attached to a first end of the double-stranded target nucleic acid molecule, and (iii) a second adapter having a double-stranded portion and a single-stranded portion attached to a second end of the double-stranded target nucleic acid molecule; (b) removing either the physically-linked nucleic acid complex amplicons from each clonal cluster bound to the surface in the (i) reverse orientation or (ii) in the forward orientation; (c) cleaving a portion of the remaining surface bound physically-linked nucleic acid complex amplicons remaining after (b) and thereby physically separating the first strand amplicons and the second strand amplicons; (d) removing the unbound physically separated first or second strand amplicons; and (e) sequencing the remaining physically separated first or second strand amplicons bound to the surface to produce a nucleic acid sequence read of the first strand or the second strand for each clonal cluster on the surface. In some aspects, cleaving at least a portion of the remaining bound physically-linked nucleic acid complex amplicons comprises preserving at least one physically-linked nucleic acid complex amplicon in at least some of the clonal clusters bound to the surface. In some aspects, the methods further comprise the steps of (f) in at least some of the clonal clusters, amplifying the at least one physically-linked nucleic acid complex amplicon on the surface to repopulate the clonal clusters of physically-linked nucleic acid complex amplicons bound to the surface; (g) removing the physically-linked nucleic acid complex amplicons that are in the other orientation from step (b); (h) removing the unbound physically separated first or second strand amplicons; (i) cleaving the remaining bound physically-linked nucleic acid complex amplicons remaining after (h) and thereby physically separating the first strand amplicons and the second strand amplicons; and (j) sequencing the remaining physically separated first or second strand amplicons bound to the surface to produce a nucleic acid sequence read of the first strand or the second strand for each clonal cluster on the surface.

In some aspects, the present disclosure provides methods of sequencing a population of double-stranded target nucleic acid molecules, each comprising a first strand and a second strand, comprising the steps of: (a) amplifying a plurality of physically-linked nucleic acid complexes bound on a surface to produce a plurality of clusters, each cluster comprising a plurality of physically-linked nucleic acid complex amplicons representing an original double-stranded target nucleic acid molecule, wherein each physically-linked nucleic acid complex amplicon comprises a first strand amplicon and a second strand amplicon, and wherein each physically-linked nucleic acid complex comprises a double-stranded target nucleic acid molecule from the population attached to (i) a first adapter comprising a linker domain between the first strand and the second strand at one end and (ii) a second adapter having a double-stranded portion and a single-stranded portion at the other end; (b) cleaving the surface bound physically-linked nucleic acid complex amplicons and thereby physically separating the first strand amplicons and the second strand amplicons; (c) removing the unbound physically separated first strand amplicons and/or the unbound physically separated second strand amplicons, wherein the remaining amplicons bound to the surface comprise (i) the physically separated first strand amplicons and (ii) the physically separated second strand amplicons; (d) sequencing the physically separated first strand amplicons bound to the surface to produce a nucleic acid sequence read of the first strand for each cluster on the surface; and (e) sequencing the physically separated second strand amplicons bound to the surface to produce a nucleic acid sequence read of the second strand for each cluster on the surface.

In some aspects, for at least some of the clusters on the surface, the methods further comprise the step of comparing the nucleic acid sequence read of the first strand to the nucleic acid sequence read of the second strand to generate an error-corrected sequence read of an original double-stranded target nucleic acid molecule. In some aspects, the methods further comprises the step of relating the nucleic acid sequence read of the first strand of an original double-stranded target nucleic acid molecule from the population to the nucleic acid sequence read of the second strand of the same original double-stranded target nucleic acid molecule using a unique molecular identifier (UMI). In some aspects, the UMI comprises a physical location on the surface. In another aspect, the UMI comprises a tag sequence, a molecule-specific feature, cluster location on the surface or a combination thereof In some aspect, the molecule-specific feature comprises nucleic acid mapping information against a reference sequence, sequence information at or near the ends of the double-stranded target nucleic acid molecule, a length of the double-stranded target nucleic acid molecule, or a combination thereof.

In some aspects, the methods further comprises the step of differentiating the nucleic acid sequence read of the first strand of an original double-stranded target nucleic acid molecule from the nucleic acid sequence read of the second strand from the same original double-stranded target nucleic acid molecule using a strand defining element (SDE). In some aspects, the SDE is the association of sequence read information with steps (e) and (j) or steps (d) and (e). In some aspects, the SDE comprises a portion of an adapter sequence.

In some aspects, sequencing the physically separated first strand amplicons or the second strand amplicons comprises sequencing by synthesis.

In some aspects, the methods further comprise the steps of preparing the physically-linked nucleic acid complexes by ligating the first adapter and the second adapter to each of a plurality of double-stranded target nucleic acid molecules in the population; and presenting the physically-linked nucleic acid complexes to the surface, the surface having a plurality of bound oligonucleotides at least partially complimentary to the single-stranded portion of the second adapters such that a plurality of physically-linked nucleic acid complexes are captured on the surface via hybridization to the plurality of bound oligonucleotides. In some aspects, the methods further comprise the step of amplifying the physically-linked nucleic acid complexes prior to the presenting step. In some aspects, amplifying the physically-linked nucleic acid complexes prior to the presenting step comprises PCR amplification or circle amplification. In other aspects, the physically-linked nucleic acid complexes are captured in both a forward and a reverse orientation on the surface.

In some aspects, the amplification step comprises bridge amplification.

In some aspects, the methods for at least some of the double-stranded target nucleic acid molecules in the population further comprise the steps of (i) comparing the sequence read from the first strand to the sequence read from the second strand; (ii) identifying a nucleotide position that does not agree between the sequence read from the first strand and the sequence read from the second strand; and (iii) generating an error-corrected sequence read of the double-stranded target nucleic acid molecule by discounting, eliminating, or correcting the identified nucleotide position that does not agree.

In some aspects, the first adapter comprises a cleavable site or motif. In some aspects, the first adapter and the second adapter each comprise a sequencing primer binding site and, optionally, a single molecule identifier (SMI) sequence. In some aspects, the second adapter comprises a sequencing primer binding site, an amplification primer binding site, an indexing sequence or any combination thereof. In some aspects, the linker domain comprises a cleavage site. In some aspects, the first adapter comprises a cleavable domain. In some aspects, the first adapter comprises a hairpin loop structure comprising a self-complementary stem portion and a single-stranded nucleotide loop portion. In some aspects, the single-stranded nucleotide loop portion comprises a cleavable domain. In some aspects, the stem portion comprises a cleavable domain. In some aspects, the cleavable domain comprises an enzyme recognition site. In some aspects, the enzyme recognition site is an endonuclease recognition site. In some aspects, the endonuclease is a restriction enzyme or a targeted endonuclease.

In some aspects, the second adapter is a “Y” shaped adapter. In some aspects, one or both arms of the Y-shaped adapter can hybridize to oligonucleotides bound to the surface.

In some aspects, the single-stranded portion of the second adapter comprises a first arm having a first primer binding site and a second arm having a second primer binding site. In some aspects, when denatured, the physically-linked double-stranded nucleic acid complex comprises from 5′ to 3′ or from 3′ to 5′: the first primer binding site, the first strand, the first adapter comprising the linker domain, the second strand, and the second primer binding site.

In some aspects, the surface is a sequencing surface. In some aspects, the surface is a flow cell. In other aspects, the surface is a surface of a bead.

In some aspects, the amplification is selected from the group consisting of PCR amplification, isothermal amplification, polony amplification, cluster amplification, and bridge amplification. In some aspects, the amplification is bridge amplification on the surface.

In some aspects, one or more of the plurality of first strand amplicons and/or the plurality of second strand amplicons is bound to the surface in a forward orientation. In some aspects, one or more of the plurality of first strand amplicons and/or the plurality of second strand amplicons is bound to the surface in a reverse orientation.

In some aspects, the methods further comprise the step of flowing the plurality of physically-linked double stranded nucleic acid complexes over the surface prior to the amplification.

In some aspects, the surface comprises a plurality of one or more bound oligonucleotides at least partially complimentary to one or more regions of the second adapter. In some aspects, the plurality of one or more bound oligonucleotides is at least partially complimentary to the single-stranded portion of the second adapter.

In some aspects, a first strand and a second strand of the physically-linked nucleic acid complex are amplified via multiple amplification reactions to generate a cluster of the physically-linked nucleic acid complex amplicons on the surface. In some aspects, the first strand and the second strand of each of the plurality of physically-linked nucleic acid complexes are amplified to generate the plurality of clusters on the surface simultaneously.

In some aspects, cleaving a portion of the bound physically-linked nucleic acid complex amplicons comprises inefficiently cleaving at a cleavable site in the first adapter resulting in both cleaved nucleic acid complexes and uncleaved nucleic acid complexes within each cluster on the surface. In some aspects, the ratio of uncleaved nucleic acid complexes of all nucleic acid complexes within each cluster on the flow cell is 1%, 5%, 10%, 20%, 30%, 40%, 45%, or 50%. In some aspects, the cleaved nucleic acid complexes are cleaved at a cleavable site in the linker domain of the first adapter by a cleavage facilitator. In some aspects, the cleavage is a site-directed enzymatic reaction. In some aspects, the cleavage facilitator is an endonuclease. In some aspects, the endonuclease is a restriction site endonuclease or a targeted endonuclease. In some aspects, the cleavage facilitator is selected from the group consisting of a ribonucleoprotein, a Cas enzyme, a Cas9-like enzyme, a meganuclease, a transcription activator-like effector-based nuclease (TALEN), a zinc-finger nuclease, an argonaute nuclease or a combination thereof. In some aspects, the cleavage facilitator comprises a CRISPR-associated enzyme. In some aspects, the cleavage facilitator comprises Cas9 or CPF1 or a derivative thereof. In other aspects, the cleavage facilitator comprises a nickase or nickase variant. In some aspects, the cleavage facilitator comprises a chemical process.

In some aspects, the amount of uncleaved nucleic acid complexes remaining on the surface can be scaled by controlling the amount or concentration of the cleavage facilitator being introduced for site-directed cleavage or by controlling the amount of time the cleavage facilitator is being introduced for site-directed cleavage. In some aspects, the uncleaved nucleic acid complexes are protected by addition of an anti-cleavage facilitator before or during the cleavage step. In some aspects, the anti-cleavage facilitator comprises an anti-cleavage motif in the linker domain of the first adapter. In some aspects, the cleavable site is already present in the linker domain of the first adapter and the anti-cleavage motif is created by hybridization of an oligonucleotide comprising an at least partially complementary sequence to the linker domain of the first adapter.

In some aspects, cleaving a portion of the bound physically-linked nucleic acid complex amplicons further comprises the steps of (i) introducing the anti-cleavage facilitator; and (ii) either following or simultaneously with (i), introducing the cleavage facilitator, wherein interaction with the anti-cleavage facilitator protects a physically-linked nucleic acid complex amplicon from cleavage. In some aspects, the cleavable site is created by hybridization of an oligonucleotide comprising an at least partially complementary sequence to the linker domain of the first adapter and wherein physically-linked nucleic acid complex amplicons not hybridized with the oligonucleotide, are not cleaved. In some aspects, the cleavable site is created by hybridization of a first oligonucleotide comprising an at least partially complementary sequence to the linker domain of the adapter and an anti-cleavage motif is created by hybridization of a second oligonucleotide comprising an at least partially complementary sequence to the linker domain of the adapter, and wherein cleaving a portion of the bound physically-linked nucleic acid complex amplicons further comprises (i) introducing a mixture of the first and second oligonucleotides; and (ii) introducing the cleavage facilitator. In some aspects, either the first oligonucleotide or the second oligonucleotide is methylated. In some aspects, the hybridization can be scaled by controlling the amount or concentration of the oligonucleotides being introduced for hybridization or by controlling the amount of time the oligonucleotides are being introduced for hybridization. In some aspects, the anti-cleavage motif comprises an oligonucleotide sequence having a bulky adduct or a side chain that prevents access to the cleavage site. In some aspects, the anti-cleavage motif comprises an oligonucleotide sequence having one or more mismatches that prevent the cleavage facilitator from recognizing the cleavage site. In some aspects, the anti-cleavage motif comprises one or more of the following: an oligonucleotide sequence having a nucleoside analogue, an abasic site, a nucleotide analogue, and a peptide-nucleic acid bond.

In some aspects, the cleaved nucleic acid complexes are cleaved at a cleavable site in the first adapter by a catalytically active enzyme and the uncleaved nucleic acid complexes are protected from cleavage in the first adapter by a catalytically inactive enzyme. In some aspects, the cleavage site is in a self-complementary portion of the first adapter or a single-stranded portion of the first adapter. In some aspects, the cleavage site is available when the physically linked nucleic acid complex amplicons are in a self-hybridized configuration on the surface. In some aspects, the cleavage site is available when the physically linked nucleic acid complex amplicons are in a double-stranded bridge amplified configuration.

In some aspects, the methods further comprise the step of selectively enriching for physically-linked nucleic acid complexes having one or more targeted genomic regions prior to step (a) to provide a plurality of enriched physically-linked nucleic acid complexes.

### BRIEF DESCRIPTION OF THE DRAWINGS

Many aspects of the present disclosure can be better understood with reference to the following figures, which together make up the Drawings. These figures are for illustration purposes only, and not for limitation. The components in the figures are not necessarily to scale. Instead, emphasis is placed on illustrating clearly the principles of the present disclosure.

FIGS. 1A and 1B are conceptual illustrations of various Duplex Sequencing method steps in accordance with an embodiment of the present technology.

FIGS. 2A and 2B illustrate nucleic acid adapter molecules for use with embodiments of the present technology and formation of double-stranded adapter-nucleic acid complexes as a result of such adapters being attached to target double-stranded nucleic acid fragments, and in accordance with another embodiment of the present technology.

FIGS. 3A-3D illustrate steps in a method for sequencing double-stranded adapter-nucleic acid complexes in accordance with an embodiment of the present technology.

FIGS. 4A-4E illustrate steps in a method for sequencing double-stranded adapter-nucleic acid complexes in accordance with another embodiment of the present technology.

FIGS. 5A-5E illustrate steps in a method for sequencing double-stranded adapter-nucleic acid complexes in accordance with a further embodiment of the present technology.

FIGS. 6-11B illustrate various adapters and use thereof in accordance with embodiments of the present technology.

FIGS. 12A-12C illustrate a method for cleaving double-stranded adapter-nucleic acid complexes in accordance with yet another embodiment of the present technology.

### DEFINITIONS

In order for the present disclosure to be more readily understood, certain terms are first defined below. Additional definitions for the following terms and other terms are set forth throughout the specification.

In this application, unless otherwise clear from context, the term “a” may be understood to mean “at least one.” As used in this application, the term “or” may be understood to mean “and/or.” In this application, the terms “comprising” and “including” may be understood to encompass itemized components or steps whether presented by themselves or together with one or more additional components or steps. Where ranges are provided herein, the endpoints are included. As used in this application, the term “comprise” and variations of the term, such as “comprising” and “comprises,” are not intended to exclude other additives, components, integers or steps.

About: The term “about”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Analog: As used herein, the term “analog” refers to a substance that shares one or more particular structural features, elements, components, or moieties with a reference substance. Typically, an “analog” shows significant structural similarity with the reference substance, for example sharing a core or consensus structure, but also differs in certain discrete ways. In some embodiments, an analog is a substance that can be generated from the reference substance, e.g., by chemical manipulation of the reference substance. In some embodiments, an analog is a substance that can be generated through performance of a synthetic process substantially similar to (e.g., sharing a plurality of steps with) one that generates the reference substance. In some embodiments, an analog is or can be generated through performance of a synthetic process different from that used to generate the reference substance.

Biological Sample: As used herein, the term “biological sample” or “sample” typically refers to a sample obtained or derived from a biological source (e.g., a tissue or organism or cell culture) of interest, as described herein. In some embodiments, a source of interest comprises an organism, such as an animal or human. In other embodiments, a source of interest comprises a microorganism, such as a bacterium, virus, protozoan, or fungus. In further embodiments, a source of interest may be a synthetic tissue, organism, cell culture, nucleic acid or other material. In yet further embodiments, a source of interest may be a plant-based organism. In yet another embodiment, a sample may be an environmental sample such as, for example, a water sample, soil sample, archeological sample, or other sample collected from a non-living source. In other embodiments, a sample may be a multi-organism sample (e.g., a mixed organism sample). In some embodiments, a biological sample is or comprises biological tissue or fluid. In some embodiments, a biological sample may be or comprise bone marrow; blood; blood cells; ascites; tissue or fine needle biopsy samples; cell containing body fluids; free floating nucleic acids; sputum; saliva; urine; cerebrospinal fluid, peritoneal fluid; pleural fluid; feces; lymph; gynecological fluids; skin swabs; vaginal swabs; pap smear, oral swabs; nasal swabs; washings or lavages such as a ductal lavages or bronchioalveolar lavages; vaginal fluid, aspirates; scrapings; bone marrow specimens; tissue biopsy specimens; fetal tissue or fluids; surgical specimens; feces, other body fluids, secretions, and/or excretions; and/or cells therefrom, etc. In some embodiments, a biological sample is or comprises cells obtained from an individual. In some embodiments, obtained cells are or include cells from an individual from whom the sample is obtained. In a particular embodiment, a biological sample is a liquid biopsy obtained from a subject. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to techniques such as amplification or reverse transcription of mRNA, isolation and/or purification of certain components, etc. Cut site: Also called “cleavage motif” and “nick site”, is the bond, or pair of bonds between nucleotides in a nucleic acid molecule. In the case of double-stranded nucleic acid molecules, such as double-stranded DNA, the cut site can entail bonds (commonly phosphodiester bonds) which are immediately adjacent from each other in a double-stranded molecule such that after cutting a “blunt” end is formed. The cut site can also entail two nucleotide bonds that are on each single strand of the pair that are not immediately opposite from each other such that when cleaved a “sticky end” is left, whereby regions of single stranded nucleotides remain at the terminal ends of the molecules. Cut sites can be defined by particular nucleotide sequence that is capable of being recognized by an enzyme, such as a restriction enzyme, or another endonuclease with sequence recognition capability such as CRISPR/Cas9. The cut site may be within the recognition sequence of such enzymes (i.e. type 1 restriction enzymes) or adjacent to them by some defined interval of nucleotides (i.e. type 2 restriction enzymes). Cut sites can also be defined by the position of modified nucleotides that are capable of being recognized by certain nucleases. For example, abasic sites can be recognized and cleaved by endonuclease VII as well as the enzyme FPG. Uracil based can be recognized and rendered into abasic sites by the enzyme UDG. Ribose-containing nucleotides in an otherwise DNA sequence can be recognized and cleaved by RNAseH2 when annealed to complementary DNA sequences.

Determine: Many methodologies described herein include a step of “determining”. Those of ordinary skill in the art, reading the present specification, will appreciate that such “determining” can utilize or be accomplished through use of any of a variety of techniques available to those skilled in the art, including for example specific techniques explicitly referred to herein. In some embodiments, determining involves manipulation of a physical sample. In some embodiments, determining involves consideration and/or manipulation of data or information, for example utilizing a computer or other processing unit adapted to perform a relevant analysis. In some embodiments, determining involves receiving relevant information and/or materials from a source. In some embodiments, determining involves comparing one or more features of a sample or entity to a comparable reference.

Duplex Sequencing (DS): As used herein, “Duplex Sequencing (DS)” is, in its broadest sense, refers to an error-correction method that achieves exceptional accuracy by comparing the sequence from both strands of individual DNA molecules.

Error-corrected: As used herein, the term “error-corrected” or “error-correction” refers to resultant products or the processes of identifying and thereafter discounting, eliminating, or otherwise correcting one or more nucleotide errors in a region of a nucleic acid molecule where two strands of a double-stranded portion of the nucleic acid molecule are not perfectly complementary to each other (e.g., due to a nucleotide mismatch). In some aspects, mismatches can be the result of a point mutation, deletion, insertion, or chemical modification. In some aspects, a mismatch includes base pairs of opposing strands with sequence, for example but not limited to, A-A, C-C, T-T, G-G, A-C, A-G, T-C, T-G, or the reverse of these pairs (which are equivalent, i.e. A-G is equivalent to G-A), a deletion, insertion, or other modification to one or more of the bases. The mismatch can be biologically-derived, DNA synthesis-derived, or a damage or modified nucleotide base caused mismatch. In some aspects, a damaged or modified nucleotide base was present on one or both strands and was converted to a mismatch by an enzymatic process (for example a DNA polymerase, a DNA glycosylase or another nucleic acid modifying enzyme or chemical process). In some aspects, this mismatch can be used to infer the presence of nucleic acid damage or nucleotide modification prior to the enzymatic process or chemical treatment.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and/or (4) post-translational modification of a polypeptide or protein.

Functionalized surface: As used herein, the term “functionalized surface” refers to a solid surface, a bead, or another fixed structure that is capable of binding or immobilizing a nucleic acid molecules or other capture moieties. In some embodiments, the functionalized surface comprises a binding moiety capable of capturing target nucleic acids. In some embodiments, a binding moiety is linked directly to a surface. In some embodiments, oligonucleotides at least partially complementary to target nucleic acids functions as the binding moiety. In some embodiments, oligonucleotides are covalently bound to the surface. In some embodiments, a functionalized surface can comprise controlled pore glass (CPG), magnetic porous glass (MPG), among other glass or non-glass surfaces. In one embodiment, a functionalized surface can be a sequencing surface, such as the surface of a flow cell. Chemical functionalization can entail ketone modification, aldehyde modification, thiol modification, azide modification, and alkyne modifications, among others. In some embodiments, the functionalized surface and an oligonucleotide used for hybridization capture are linked using one or more of a group of immobilization chemistries that form amide bonds, alkylamine bonds, thiourea bonds, diazo bonds, hydrazine bonds, among other surface chemistries. In some embodiments, the functionalized surface and an oligonucleotide used for hybridization capture are linked using one or more of a group of reagents including EDAC, NHS, sodium periodate, glutaraldehyde, pyridyl disulfides, nitrous acid, biotin, among other linking reagents.

gRNA: As used herein, “gRNA” or “guide RNA”, refers to short RNA molecules which include a scaffold sequence suitable for a targeted endonuclease (e.g., a Cas enzyme such as Cas9 or Cpf1 or another ribonucleoprotein with similar properties, etc.) binding to a substantially target-specific sequence which facilitates cutting of a specific region of DNA or RNA.

Mutation: As used herein, the term “mutation” refers to alterations to nucleic acid sequence or structure relative to a reference sequence. Mutations to a polynucleotide sequence can include point mutations (e.g., single base mutations), multi-nucleotide mutations, nucleotide deletions, sequence rearrangements, nucleotide insertions, and duplications of the DNA sequence in the sample, among complex multi-nucleotide changes. Mutations can occur on both strands of a duplex DNA molecule as complementary base changes (i.e. true mutations), or as a mutation on one strand but not the other strand (i.e. heteroduplex), that has the potential to be either repaired, destroyed or be mis-repaired/converted into a true double-stranded mutation. Reference sequences may be present in databases (i.e. HG38 human reference genome) or the sequence of another sample to which a sequence is being compared. Mutations are also known as genetic variant.

Nucleic acid: As used herein, in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to an individual nucleic acid residue (e.g., a nucleotide and/or nucleoside); in some embodiments, “nucleic acid” refers to an oligonucleotide chain comprising individual nucleic acid residues. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. For example, in some embodiments, a nucleic acid is, comprises, or consists of one or more “peptide nucleic acids”, which are known in the art and have peptide bonds instead of phosphodiester bonds in the backbone, are considered within the scope of the present technology. Alternatively, or additionally, in some embodiments, a nucleic acid has one or more phosphorothioate and/or 5′-N-phosphoramidite linkages rather than phosphodiester bonds. In some embodiments, a nucleic acid is, comprises, or consists of one or more natural nucleosides (e.g., adenosine, thymidine, guanosine, cytidine, uridine, deoxyadenosine, deoxythymidine, deoxy guanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a nucleic acid comprises one or more modified sugars (e.g., 2′-fluororibose, ribose, 2′-deoxyribose, arabinose, hexose or Locked Nucleic acids) as compared with those in commonly occurring natural nucleic acids. In some embodiments, a nucleic acid has a nucleotide sequence that encodes a functional gene product such as an RNA or protein. In some embodiments, a nucleic acid includes one or more introns. In some embodiments, a nucleic acid may be a non-protein coding RNA product, such as a microRNA, a ribosomal RNA, or a CRISPR/Cas9 guide RNA. In some embodiments, a nucleic acid serves a regulatory purpose in a genome. In some embodiments, a nucleic acid does not arise from a genome. In some embodiments, a nucleic acid includes intergenic sequences. In some embodiments, a nucleic acid derives from an extrachromosomal element or a nonnuclear genome (mitochondrial, chloroplast etc.), In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 2, 3, 4, 5, 6, 7, 8, 9, 10, 15, 20, 25, 30, 35, 40, 45, 50, 55, 60, 65, 70, 75, 80, 85, 90, 95, 100, 1 10, 120, 130, 140, 150, 160, 170, 180, 190, 200, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a nucleic acid is partly or wholly single stranded; in some embodiments, a nucleic acid is partly or wholly double-stranded. In some embodiments a nucleic acid has a nucleotide sequence comprising at least one element that encodes, or is the complement of a sequence that encodes, a polypeptide. In some embodiments, a nucleic acid has enzymatic activity. In some embodiments the nucleic acid serves a mechanical function, for example in a ribonucleoprotein complex or a transfer RNA. In some embodiments a nucleic acid function as an aptamer. In some embodiments a nucleic acid may be used for data storage. In some embodiments a nucleic acid may be chemically synthesized in vitro.

Reference: As used herein describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Sequence read: As used herein, the term “sequence read” or “sequencing read” refers to nucleic acid sequence data corresponding to a reference or target nucleic acid molecule. In some aspects, the data is an inferred sequence of base pairs (or base pair probabilities) corresponding to all or part of (e.g., a fragment or portion of) the reference or target nucleic acid molecule processed by a sequencing platform. Sequence read lengths can range from several base pairs (bp) to hundreds of kilobases (kb). Sequence read lengths can be impacted by the size or length of the reference or target nucleic acid molecule and the sequencing platform used. In some aspects, the sequence read is generated using sequencing technologies such as but not limited to, next generation sequencing platforms, e.g., Illumina® HiSeq® Illumina® NovaSeq®, Illumina® NextSeq®, Illumina® MiSeq®, Illumina® iSeq®, Oxford Nanopore sequencing systems, ThermoFisher® Ion Torrent® sequencing systems, Roche 454 GS System®, Illumina Genome Analyzer®, Applied Biosystems SOLiD System®, Helicos Heliscope®, Complete Genomics®, and Pacific Biosciences SMRT®.

Single Molecule Identifier (SMI): As used herein, the term “single molecule identifier” or “SMI”, (which may be referred to as a “tag” a “barcode”, a “Molecular bar code”, a “Unique Molecular Identifier”, or “UMI”, among other names) refers to any material (e.g., a nucleotide sequence, a nucleic acid molecule feature) that is capable of distinguishing an individual molecule in a large heterogeneous population of molecules. In some embodiments, a SMI can be or comprise an exogenously applied SMI. In some embodiments, an exogenously applied SMI may be or comprise a degenerate or semi-degenerate sequence. In some embodiments substantially degenerate SMIs may be known as Random Unique Molecular Identifiers (R-UMIs). In some embodiments an SMI may comprise a code (for example a nucleic acid sequence) from within a pool of known codes. In some embodiments pre-defined SMI codes are known as Defined Unique Molecular Identifiers (DUMIs). In some embodiments, a SMI can be or comprise an endogenous SMI. In some embodiments, an endogenous SMI may be or comprise information related to specific shear-points of a target sequence, or features relating to the terminal ends of individual molecules comprising a target sequence. In some embodiments an SMI may relate to a sequence variation in a nucleic acid molecule cause by random or semirandom damage, chemical modification, enzymatic modification or other modification to the nucleic acid molecule. In some embodiments the modification may be deamination of methylcytosine. In some embodiments the modification may entail sites of nucleic acid nicks. In some embodiments, an SMI may comprise both exogenous and endogenous elements. In some embodiments an SMI may comprise physically adjacent SMI elements. In some embodiments SMI elements may be spatially distinct in a molecule. In some embodiments an SMI may be a non-nucleic acid. In some embodiments an SMI may comprise two or more different types of SMI information. Various embodiments of SMIs are further disclosed in International Patent Publication No. WO2017/100441, which is incorporated by reference herein in its entirety.

Strand Defining E1ement (SDE): As used herein, the term “Strand Defining Element” or “SDE”, refers to any material which allows for the identification of a specific strand of a double-stranded nucleic acid material and thus differentiation from the other/complementary strand (e.g., any material that renders the amplification products of each of the two single stranded nucleic acids resulting from a target double-stranded nucleic acid substantially distinguishable from each other after sequencing or other nucleic acid interrogation). In some embodiments, a SDE may be or comprise one or more segments of substantially non-complementary sequence within an adapter sequence. In particular embodiments, a segment of substantially noncomplementary sequence within an adapter sequence can be provided by an adapter molecule comprising a Yshape or a “loop” shape. In other embodiments, a segment of substantially non-complementary sequence within an adapter sequence may form an unpaired “bubble” in the middle of adjacent complementary sequences within an adapter sequence. In other embodiments an SDE may encompass a nucleic acid modification. In some embodiments an SDE may comprise physical separation of paired strands into physically separated reaction compartments. In some embodiments an SDE may comprise a chemical modification. In some embodiments an SDE may comprise a modified nucleic acid. In some embodiments an SDE may relate to a sequence variation in a nucleic acid molecule caused by random or semi-random damage, chemical modification, enzymatic modification or other modification to the nucleic acid molecule. In some embodiments the modification may be deamination of methylcytosine. In some embodiments the modification may entail sites of nucleic acid nicks. Various embodiments of SDEs are further disclosed in International Patent Publication No. WO2017/100441, which is incorporated by reference herein in its entirety.

Subject: As used herein, the term “subject” refers an organism, typically a mammal (e.g., a human, in some embodiments including prenatal human forms). In some embodiments, a subject is suffering from a relevant disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Variant: As used herein, the term “variant” refers to an entity that shows significant structural identity with a reference entity, but differs structurally from the reference entity in the presence or level of one or more chemical moieties as compared with the reference entity. In the context of nucleic acids, a variant nucleic acid may have a characteristic sequence element comprised of a plurality of nucleotide residues having designated positions relative to another nucleic acid in linear or three-dimensional space. Sequences with homology differ by one or more variant. For example, a variant polynucleotide (e.g., DNA) may differ from a reference polynucleotide as a result of one or more differences in nucleic acid sequence. In some embodiments, a variant polynucleotide sequence includes an insertion, deletion, substitution or mutation relative to another sequence (e.g., a reference sequence or other polynucleotide (e.g., DNA) sequences in a sample). Examples of variants include SNPs, SNVs, CNVs, CNPs, MNVs, MNPs., mutations, cancer mutations, driver mutations, passenger mutations, inherited polymorphisms.

### DETAILED DESCRIPTION

The present technology relates generally to methods for providing error-corrected sequence reads for nucleic acid material using Duplex Sequencing and associated reagents for use in such methods. Some embodiments of the technology are directed to methods for achieving high accuracy sequencing reads that is provided at a faster rate (e.g., with fewer steps) and/or with less cost (e.g., utilizing fewer reagents), and resulting in increased desirable data. Other aspects of the technology are directed to methods and reagents for increasing conversion efficiency (i.e., proportion of nucleic acid molecules for which sequences are produced) for Duplex Sequencing. Various aspects of the present technology have many applications in both pre-clinical and clinical testing and diagnostics as well as other applications.

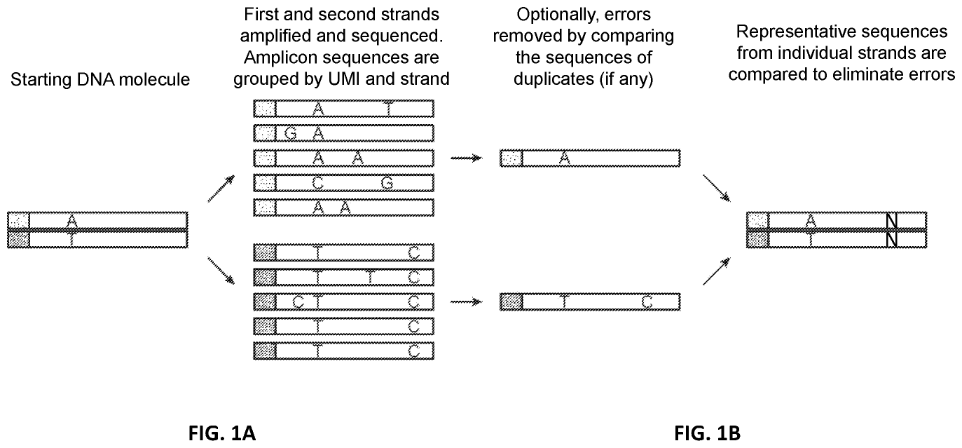

Specific details of several embodiments of the technology are described below and with reference to the FIGS. 1A-12C. Although many of the embodiments are described herein with respect to Duplex Sequencing, other sequencing modalities capable of generating error-corrected sequencing reads and other sequencing modalities for providing sequence information in addition to those described herein are within the scope of the present technology. Further, other embodiments of the present technology can have different configurations, components, or procedures than those described herein. A person of ordinary skill in the art, therefore, will accordingly understand that the technology can have other embodiments with additional elements and that the technology can have other embodiments without several of the features shown and described below with reference to the FIGS. 1A-12C.

With regard to the efficiency of a Duplex Sequencing process or other high-accuracy sequencing modality, conversion efficiency can be defined as the fraction of unique nucleic acid molecules inputted into a sequencing library preparation reaction from which at least one duplex consensus sequence read (or other high-accuracy sequence read) is produced. In some instances, conversion efficiency shortcomings may limit the utility of high-accuracy Duplex Sequencing for some applications where it would otherwise be very well suited. For example, a low conversion efficiency would result in a situation where the number of copies of a target double-stranded nucleic acid is limited, which may result in a less than desired amount of sequence information produced. Non-limiting examples of this concept include DNA from circulating tumor cells or cell-free DNA derived from tumors, or prenatal infants that are shed into body fluids such as plasma and intermixed with an excess of DNA from other tissues. Other non-limiting examples includes forensic material, such as that left at a crime scene in limited amounts, ancient DNA, such as may be found at an archeological site, very small biopsies, such as those obtained with a needle biopsy, aspirate or endoscopically, small amounts of formalin-fixed clinical material, samples that have been micro-dissected, samples from small biological regions or human or non-human organisms, samples or hair, blood spots or other biological material produced by, or originating from a multicellular organism or single cell organism in limited quantities, including single cells or small numbers of cells. Although Duplex Sequencing typically has the accuracy to be able to resolve one mutant molecule among more than one hundred thousand unmutated molecules, if only 10,000 molecules (e.g. 10,000 genome-equivalents in the case of single copy genes or loci) are available in a sample, for example, and even with the ideal efficiency of converting these to duplex consensus sequence reads being 100%, the lowest mutation frequency that could be measured would be 1/(10,000*100%)=1/10,000. As a clinical diagnostic, having maximum sensitivity to detect the low-level signal of a cancer or a therapeutically or diagnostically-relevant mutation can be important and so a relatively low conversion efficiency would be undesirable in this context. Similarly, in forensic applications, often very little DNA is available for testing. When only nanogram or picogram quantities can be recovered from a crime scene or site of a natural disaster, and/or where the DNA from multiple individuals is mixed together, having maximum conversion efficiency can be important in being able to detect the presence of the DNA of all individuals within the mixture.

Methods incorporating Duplex Sequencing, as well as other sequencing modalities, may include attachment (e.g., ligation) of one or more sequencing adapters to a target double-stranded nucleic acid molecule to produce a double-stranded target nucleic acid complex. Such adapter molecules may include one or more of a variety of features suitable for massive parallel sequencing platforms such as, for example, sequencing primer recognition sites, amplification primer recognition sites, barcodes (e.g., single molecule identifier (SMI)) sequences (also known as unique molecular identifier (UMI)), indexing sequences, single-stranded portions, double-stranded portions, strand distinguishing elements or features, and the like. As discussed above, to obtain Duplex Sequencing information, successful recovery of sequence information from both strands of the original duplex molecules is needed. Aspects of the present disclosure provide methods and reagents for generating and associating sequencing information from both strands of the original duplex molecules via physically linking the strands before amplification and sequencing.

I. Selected Embodiments of Duplex Sequencing Methods and Associated Adapters and Reagents

Duplex Sequencing is a method for producing error-corrected DNA sequences from double-stranded nucleic acid molecules and was originally described in International Patent Publication No. WO 2013/142389 and in U.S. Pat. No. 9,752,188, both of which are incorporated herein by reference in their entireties. In certain aspects of the technology, Duplex Sequencing can be used to sequence both strands of individual DNA molecules in such a way that the derivative sequence reads can be recognized as having originated from the same double-stranded nucleic acid parent molecule during massively parallel sequencing (MPS), also commonly known as next generation sequencing (NGS), but also differentiated from each other as distinguishable entities following sequencing. The resulting sequence reads from each strand are then compared for the purpose of obtaining an error-corrected sequence of the original double-stranded nucleic acid molecule.

FIG. 1 is a conceptual illustration of various Duplex Sequencing method steps in accordance with an embodiment of the present technology. In certain embodiments, methods incorporating Duplex Sequencing may include ligation of one or more sequencing adapters to a plurality of target double-stranded nucleic acid molecules each comprising a first strand target nucleic acid sequence and a second strand target nucleic sequence to produce a plurality of double-stranded target nucleic acid complexes (FIG. 1A). Once preparation of a double-stranded nucleic acid library is formed, the complexes can be subjected to DNA amplification, such as with PCR, or any other biochemical method of DNA amplification (e.g., rolling circle amplification, multiple displacement amplification, isothermal amplification, bridge amplification, polony amplification, isothermal amplification or surface-bound amplification, such that one or more copies of the first strand target nucleic acid sequence and one or more copies of the second strand target nucleic acid sequence are produced (e.g., FIG. 1A). The one or more amplification copies of the first strand target nucleic acid molecule and the one or more amplification copies of the second target nucleic acid molecule can then be subjected to DNA sequencing, preferably using a “Next-Generation” massively parallel DNA sequencing platform (e.g., FIG. 1A).

Following sequencing, a sequence read produced from the first strand of the target nucleic acid molecule is compared to a sequence read produced from the second strand of the same target nucleic acid molecule. In some embodiments, more than one sequence read can be generated from the first and second strands. Once compared, an error-corrected target nucleic acid molecule sequence can be generated (e.g., FIG. 1B). For example, nucleotide positions where the bases from both the first and second strand target nucleic acid sequences agree are deemed to be true sequences, whereas nucleotide positions that disagree between the two strands are recognized as potential sites of technical errors that may be discounted, eliminated, corrected or otherwise identified. In some embodiments, when nucleotide positions disagree, the site can be identified as unknown (e.g., shown as “N” in FIG. 1B). An error-corrected sequence of the original double-stranded target nucleic acid molecule can thus be produced (shown in FIG. 1B). Optionally, and in some embodiments, and following separately grouping of each of the sequencing reads produced from the first strand target nucleic acid molecule and the second strand target nucleic acid molecule, a single-strand consensus sequence can be generated for each of the first and second strands. The single-stranded consensus sequences from the first strand target nucleic acid molecule and the second strand target nucleic acid molecule can then be compared to produce an error-corrected target nucleic acid molecule sequence (e.g., FIG. 1B).

Alternatively, in some embodiments, sites of sequence disagreement between the two strands can be recognized as potential sites of biologically-derived mismatches in the original double-stranded target nucleic acid molecule. Alternatively, in some embodiments, sites of sequence disagreement between the two strands can be recognized as potential sites of DNA synthesis-derived mismatches in the original double-stranded target nucleic acid molecule. Alternatively, in some embodiments, sites of sequence disagreement between the two strands can be recognized as potential sites where a damaged or modified nucleotide base was present on one or both strands and was converted to a mismatch by an enzymatic process (for example a DNA polymerase, a DNA glycosylase or another nucleic acid modifying enzyme or chemical process). In some embodiments the modified nucleotide base is 5-methyl-cytosone, 8-oxo-guanine, a ribose base, an abasic nucleotide, or a uracil nucleotide. In some embodiments, this latter finding can be used to infer the presence of nucleic acid damage or nucleotide modification prior to the enzymatic process or chemical treatment.

In certain embodiments, and as described in U.S. Pat. No. 9,752,188 and International Patent Publication No. WO 2017/100441, first strand sequencing reads and second strand sequencing reads from an individual original double-stranded nucleic acid molecule can be associated (e.g., grouped) using (a) single molecular identifier (SMI) sequences associated with the adapters during library preparation; (b) fragment features associated with the original double-stranded molecule, such as sequences at or near or relative to fragment ends; and (c) combinations thereof.

In one embodiment, generation of raw sequence reads for use in Duplex Sequencing embodies the use of a target double-stranded nucleic acid molecule with a hairpin adapter attached to one end of the molecule, and a “Y” shaped adapter attached to the other end of the molecule. This linked or two-stranded complex comprising both a first strand and a second strand of the original double-stranded nucleic acid molecule can further be amplified using any type of amplification (for example, PCR or bridge), and can then undergo massively parallel sequencing (for example, sequencing by synthesis, Next Generation Sequencing (NGS), etc.), in order to generate sequence reads for use in Duplex Sequencing. Adapter-double-stranded nucleic acid complexes with hairpin adapters (i.e. “loop” or “U” shape) allow for, in a non-limiting example, the generation of sequence reads from both the original first strand and the original second strand of the target double-stranded nucleic acid molecules in a manner that allows the sequence reads to be grouped by nature of the location of the sequencing reaction on a flow cell surface (if doing sequencing by synthesis) or otherwise in the location of the sequencing reaction/process.

Aspects of the present technology are directed to methods and reagents for associating and/or grouping first and second strand sequencing reads by physically linking first and second strands in a manner such that sequencing information derived from both strands are associated with each other (e.g., for error correction) by nature of their physical linkage. In certain embodiments, methods for preparing a sequencing library for use in Duplex Sequencing may include the ligation of a hairpin adapter to one end of a target double-stranded nucleic acid molecule, and the ligation of a “Y” shaped adapter to the opposite end of the same target double-stranded nucleic acid molecule. In one embodiment, the hairpin adapter molecules comprise a cleavable hairpin adapter element for targeted separation of first and second strands of the target double-stranded nucleic acid molecule.

In some embodiments, association of first strand sequence reads and second strand sequencing reads can be accomplished during or following sequencing reactions on a sequencer. For example, in certain embodiments, first and second strands of the double-stranded nucleic acid molecule are linked by an intervening linker domain, such as for example, a hairpin adapter sequence. In one embodiment, sequence information derived from both of the strands of the original nucleic acid molecule are generated within the same clonal cluster on a MPS sequencer (e.g., on a flow cell). Challenges to sequencing linked first and second strands on a sequencer occur because self-complementary hairpin sequences can preferentially hybridize on the sequencing surface or in solution, impairing polymerase extension. Certain aspects of the present technology disclose methods for overcoming these challenges associated with self-complementary hybridization of linked first and second strands while being able to obtain sequencing reads from both the first and second strands within the same clonal cluster on the sequencer.

Adapters and Adapter Sequences

In various arrangements, adapter molecules that comprise primer sites, flow cell sequences and/or other features, such as SMIs (e.g., molecular barcodes) or SDEs, are contemplated for use with many of the embodiments disclosed herein. In some embodiments, provided adapters may be or comprise one or more sequences complimentary or at least partially complimentary to PCR primers (e.g., primer sites) that have at least one of the following properties: 1) high target specificity; 2) capable of being multiplexed; and 3) exhibit robust and minimally biased amplification.

In some embodiments, adapter molecules can be “Y”-shaped, “U”-shaped, “hairpin” shaped, have a bubble (e.g., a portion of sequence that is non-complimentary), or other features. In other embodiments, adapter molecules can comprise a “Y”-shape, a “U”-shape, a “hairpin” shape, or a bubble. For the purposes of this disclosure a “U”-shaped or “hairpin” shaped adapter may both be used to collectively refer to an adapter with a linker domain that links or connects a first strand of a target double-stranded nucleic acid molecule to a second strand of the same molecule. Certain adapters may comprise modified or non-standard nucleotides, restriction sites, or other features for manipulation of structure or function in vitro. Adapter molecules may ligate to a variety of nucleic acid material having a terminal end. For example, adapter molecules can be suited to ligate to a T-overhang, an A-overhang, a CG-overhang, a multiple nucleotide overhang (also referred to herein as a “sticky end” or “sticky overhang”) or single-stranded overhang region with known nucleotide length (e.g., 1, 2, 3, 4, 5, 6, 7, 8, 9, 10, 11, 12, 13, 14, 15, 16, 17, 18, 19, 20 or more nucleotides), a dehydroxylated base, a blunt end of a nucleic acid material and the end of a molecule were the 5′ of the target is dephosphorylated or otherwise blocked from traditional ligation. In other embodiments the adapter molecule can contain a dephosphorylated or otherwise ligation-preventing modification on the 5′ strand at the ligation site. In the latter two embodiments such strategies may be useful for preventing dimerization of library fragments or adapter molecules.

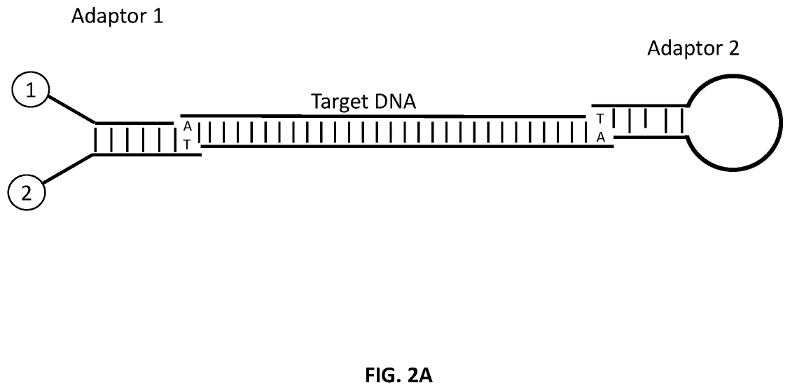

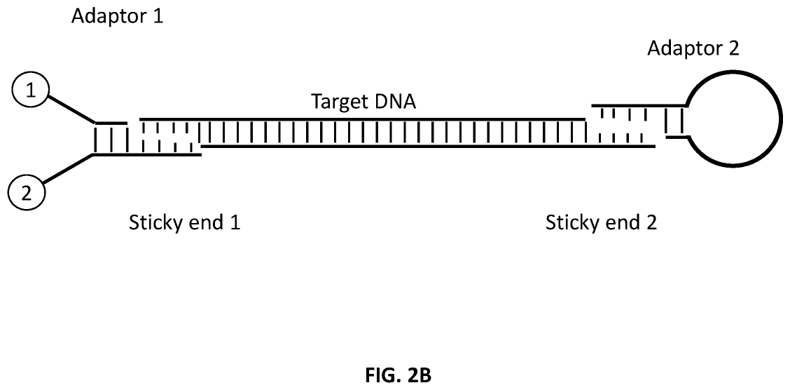

FIG. 2A illustrates nucleic acid adapter molecules for use with some embodiments of the present technology and a double-stranded adapter-nucleic acid complex resulting from ligation of the adapter molecules to a double-stranded nucleic acid fragment in accordance with an embodiment of the present technology. As shown in FIG. 2A, a first adapter molecule (Adapter 1) can be a Y-shaped adapter molecule having first and second primer sites (labelled as primer site 1 and primer site 2) and suitable for ligation to the double-stranded nucleic acid fragment by way of a T-overhang. A second adapter molecule (Adapter 2) suitable for ligation to the target nucleic acid fragment by way of a T-overhang is shown as a hairpin adapter comprising a single-stranded linkage domain. Sequencing library generation of a population of double-stranded nucleic acid fragments can include ligating a pool of adapters comprising both Adapter 1 and Adapter 2 to the population of double-stranded nucleic acid fragments. FIG. 2A illustrates one resultant product of this described ligation reaction. Other products would include adapter-nucleic acid complexes comprising Adapter 1 at both ends and adapter-nucleic acid complexes comprising Adapter 2 at both ends. In various embodiments described herein, it is desirable to generate the adapter-nucleic acid complex as illustrated in FIG. 2A for use with Duplex Sequencing methods.

FIG. 2B illustrates another embodiment, wherein the target double-stranded nucleic acid fragments comprise a sticky end 1 at one end of the fragment and a sticky end 2 at the opposite end of the fragment. By design the sequence of sticky end 1 (overhang at the 5′ end of the targeted fragment) is known. Likewise, the sequence of sticky end 2 (overhang at the 3′ end of the targeted fragment) is known. In one embodiment, the sequence of sticky end 1 is different than the sequence of sticky end 2. In another embodiment, the sequence of sticky end 1 is a different length than the sequence of sticky end 2. In a further embodiment, sticky end 1 is a 5′ overhang and sticky end 2 is a 3′ overhang. Specific adapters comprising substantially complementary sequences can be synthesized such that fragments can be attached to adapters at both ends. In one embodiment, the adapters can be different (e.g., adapter 1 can comprise a Y-shape and adapter 2 can comprise a U-shape). In other embodiments (not shown) the adapters can be the same type of adapters (e.g., adapters comprising a Y-shape, U-shape, barcoded adapters, etc.). As illustrated in FIG. 2B, this design allows for each target double-stranded nucleic acid molecule to have a Y-shaped adapter on one end and a hairpin (e.g., adapter with linkage domain) on the other end. As such, when denatured, the adapter-nucleic acid complex comprises a single-stranded molecule comprising a first primer site, a first strand, a linkage domain, a second strand, and a second primer site. There may be advantages in other applications to designing specific adapters to be positioned in either the 5′ or 3′ ends of fragments. The specificity of substantially unique sticky ends on the targeted fragments facilitates these types of applications. Moreover, positive selection of successfully cut and adapter ligated target fragments can ensure only amplification and sequencing of the target enriched nucleic acid regions.

Accordingly, in some embodiments, sets of adapter molecules may comprise different or unique or semi-unique sticky overhangs with respect to other sets of adapter molecules. The number of different types of sticky ends may be 2 or 3, 4, 5, 6, 7, 8, 9 or 10 or more. It may be about 11 or 12 or 15 or 20 or 25 or 30 or 35 or 40 or 45 or 50 or 60 or 70 or 80 or 90 or 100 or 120 or 140 or 150 or 200 or 300 or 400 or 500 or 750 or 1000 or more. In a particular example, a hairpin adapter molecule can comprise a first sticky overhang suitable to ligate to a first, complementary fragment sticky end, and a Y-shaped adapter can comprise a second sticky overhand suitable to ligate to a second, complementary fragment sticky end. As such, sequencing library preparation of a population of nucleic acid molecules can comprise generating nucleic acid fragments having a first sticky end and a second sticky end and ligating the nucleic acid fragments to the hairpin and Y-shaped adapters. Resultant sequencing library can comprise a plurality of double-stranded adapter- nucleic acid fragment complexes each having a hairpin adapter on a first end and a Y-shaped adapter on a second end.

Amplification

In one embodiment, the method can include amplification of adapter-nucleic acid complexes comprising both the first and second strands on a sequencer surface, such as the surface of a flow cell. In some embodiments, amplification on a surface, such as bridge amplification on a surface of a flow cell, includes generating clusters or multiple of copies of bound nucleic acid template. In a particular embodiment, linked first and second strand nucleic acid templates can bridge amplify on the surface of a flow cell, for example, to generate a plurality of clonal clusters, wherein each clonal cluster comprises nucleic acid template copies derived from both the original first and second strands of the original double-stranded nucleic acid molecule. Some of the clonal copies in a cluster will be in the forward orientation, while the rest will be in the reverse orientation. One of ordinary skill in the art will appreciate various embodiments for polony amplification, cluster amplification, bridge amplification and the like using amplification, including steps of flowing the adapter-nucleic acid complexes over a surface providing bound oligonucleotides at least partially complimentary to regions of the Y-shaped adapter. A surface can be provided with one or more than one oligonucleotide complementary to portion(s) of the adapter(s). In practice, both arms of the Y-shaped adapter can hybridize to the surface of the flow cell.

Bridge amplification (not shown) can be used to generate multiple copies of the complexes to form a colony or cluster (also referred to as a clonal cluster herein). Each clonal cluster comprises the multiple copies derived from an original molecule (e.g., an adapter-nucleic acid complex) in both the forward orientation and the reverse orientation.

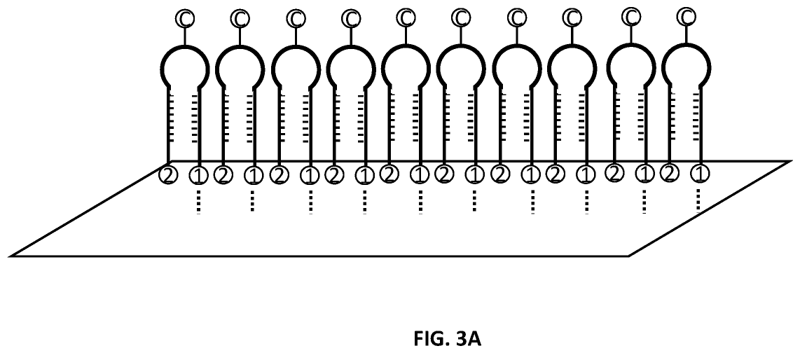

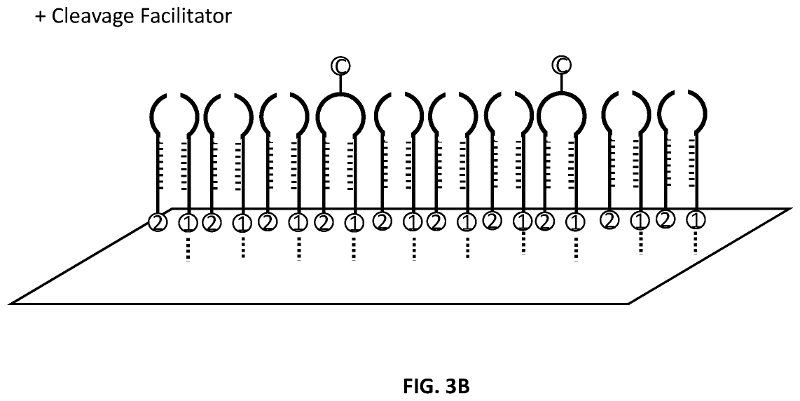

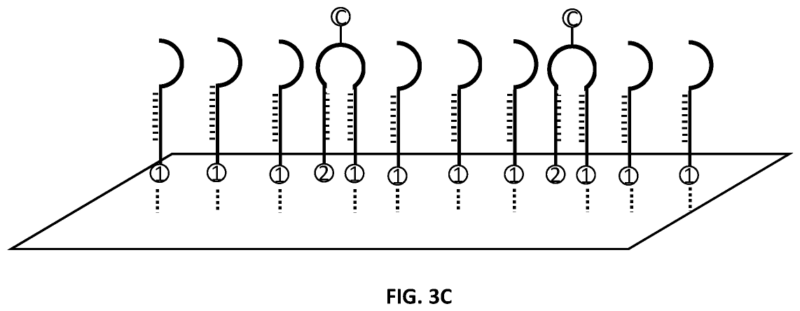

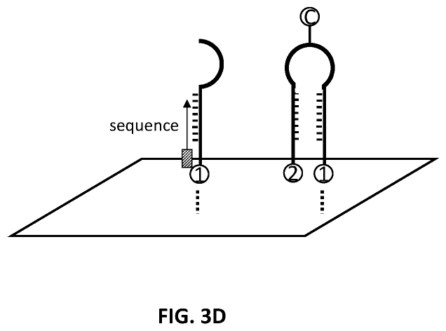

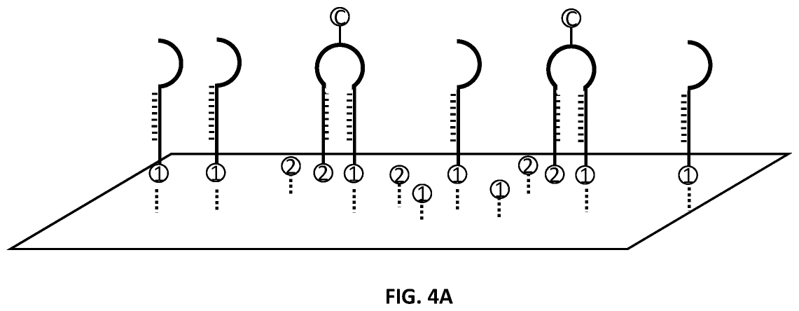

In one embodiment, a sequencing reaction can proceed when either the copies in the forward orientation or the copies in the reverse orientation is cleaved and removed. FIG. 3A illustrates a step in the process after bridge amplification of an adapter-nucleic acid complex (e.g., a two-stranded nucleic acid complex) and after copies comprising the forward orientation (e.g., wherein nucleic acid sequence “2” is bound to the surface of the flow cell) are cut and removed. As shown in FIG. 3A, the remaining complexes are in the reverse orientation (e.g., wherein nucleic acid sequence “1” is bound to the surface of the flow cell; e.g., the 3′ end of the molecule is bound to the surface). In one embodiment, the nucleic acid sequence of the first strand readily hybridizes with the complementary nucleic acid sequence of the second strand making sequencing by synthesis of the longer complex difficult. The bound copies of the illustrated complex comprise a linker domain as provided by the hairpin adapter (e.g., Adapter 2, FIGS. 2A and 2B). In some embodiments, the linker domain comprises a cleavable site or motif (“C”). The cleavable site C may comprise a nucleotide sequence, a single nucleotide base, a modified base, or other enzymatically or non-enzymatically cleavable feature.

As shown in FIG. 3B, the process can include a step comprising cleavage of the cleavable site C to separate the first strand sequence from the second strand sequence. In one embodiment, the cleavage event at site C can be facilitated by a cleavage facilitator (e.g., an enzyme, a chemical, etc.). In one embodiment, the cleavage step can be inefficient such that only a portion of the complexes are cleaved at the site C. As such, a portion (e.g., about 1%, about 2%, about 3%, about 4%, about 5%, about 6%, about 7%, about 8%, about 9%, about 10%, about 15%, about 20%, about 25%, about 30%, about 40%, about 45%, about 50% or more or less; about 1% to about 10%; about 10% to about 25%, about 25% to about 45%; greater than 50%, less than 10%, etc.) of the complexes can remain uncleaved and the first and second strand sequences remain linked. In some aspects, at least 50%, at least 60%, at least 70%, at least 80%, at least 90%, at least 91%, at least 92%, at least 93%, at least 94%, at least 95%, at least 96%, at least 97%, at least 98%, at least 99%, or 100% of the complexes are cleaved, e.g., at the site C.

Upon separation of the first strand from the second strand by cleavage at site C, the unbound strand (e.g., proximate nucleic acid sequence 2), will be washed away. For example, as shown in FIG. 3C, the portion of complexes that were cleaved at site C comprise only the nucleotide sequence of the first strand and a portion of the hairpin adapter. Because the complex will no longer self-hybridize, a sequencing reaction using a primer specific to the adapter (e.g., at or near nucleotide sequence 1, the 3′ end of the bound molecule) can be used to perform a sequencing reaction for generating a sequencing read of the first strand remaining in the clonal cluster (FIG. 3D). Indexing reads can also be generated (not shown). Note that the sequencing read of the first strand is a single-end sequence read. The complexes that remain uncleaved in the clonal cluster remain self-hybridized and will most likely not successfully sequence during the sequencing reaction due to the difficulty of displacement of the longer second strand by the sequencing primer (FIG. 3D).

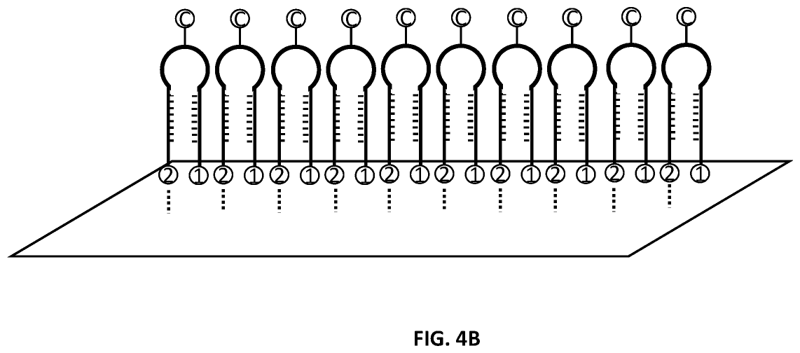

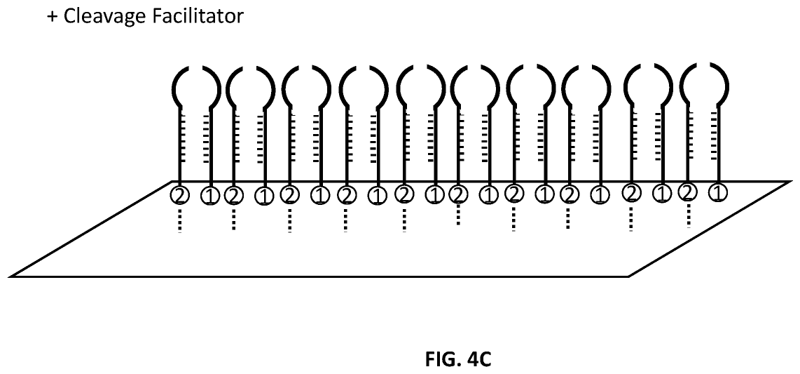

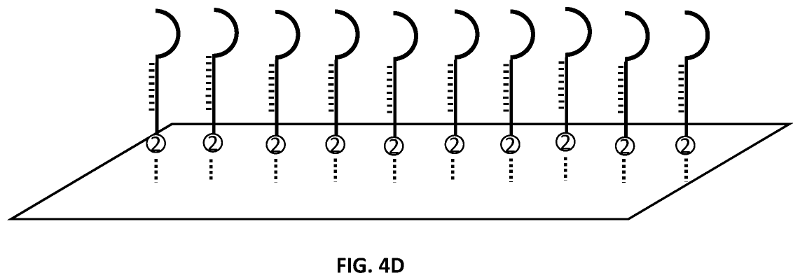

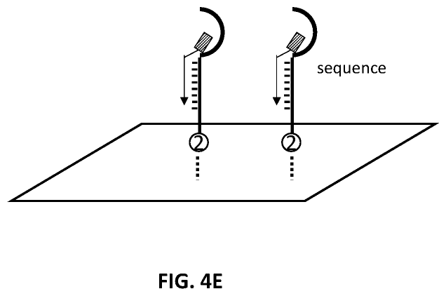

After obtaining sequencing information from the first strand present in the clonal cluster, a next step in the process comprises a second round of amplification (e.g., bridge amplification) to provide more copies of the uncleaved complexes. Bridge amplification requires the presence of both nucleic acid sequence 1 and nucleic acid sequence 2 that is present on the full-length complexes. Only the remaining uncleaved complexes have both adapter sequences still present. As such, the clonal cluster can be repopulated by bridge amplification utilizing remaining oligonucleotides bound to the surface of the flow cell (FIG. 4A).

Following amplification, a second sequencing reaction can proceed when either the copies in the reverse orientation is cleaved and removed. FIG. 4B illustrates a step in the process after bridge amplification of an adapter-nucleic acid complex (e.g., a two-stranded nucleic acid complex) and after copies comprising the reverse orientation (e.g., wherein nucleic acid sequence “1” is bound to the surface of the flow cell) are cut and removed. As shown in FIG. 4B, the remaining complexes are in the forward orientation (e.g., wherein nucleic acid sequence “2” is bound to the surface of the flow cell; e.g., wherein the 5′ end of the molecule is bound to the surface). As described above, the nucleic acid sequence of the first and second strands readily hybridize making sequencing by synthesis of the longer complex difficult.

As shown in FIG. 4C, the process can include a step comprising cleavage of the cleavable site C to separate the second strand sequence from the first strand sequence. In one embodiment, the cleavage event at site C can be facilitated by a cleavage facilitator (e.g., an enzyme, a chemical, etc.). As discussed above, the cleavage step can be inefficient such that only a portion of the complexes are cleaved and the site C. As such, a portion (e.g., about 1%, about 2%, about 3%, about 4%, about 5%, about 6%, about 7%, about 8%, about 9%, about 10%, about 15%, about 20%, about 25%, about 30%, about 40%, about 45%, about 50% or more or less; about 1% to about 10%; about 10% to about 25%, about 25% to about 45%; greater than 50%, less than 10%, etc.) of the complexes can remain uncleaved and the first and second strand sequences remain linked. Alternatively, the cleavage step can be efficient, and all complexes can be cleaved (e.g., as illustrated in FIG. 4C)

Upon separation of the second strand from the first strand by cleavage at site C, the unbound strand (e.g., proximate nucleic acid sequence 1), will be washed away. For example, as shown in FIG. 4D, the portion of complexes that were cleaved at site C comprise only the nucleotide sequence of the second strand and a portion of the hairpin adapter. Because the complex will no longer self-hybridize, a sequencing reaction using a primer specific to the remaining portion of the hairpin adapter can be used to perform a sequencing reaction for generating a sequencing read of the second strand remaining in the clonal cluster (FIG. 4E). Indexing reads can also be generated (not shown). Note that the sequencing read of the second strand is a single-end sequence read. Once sequence reads derived from both the first and second strands (e.g., within the same clonal cluster) are generated, they can be compared for error-correction.

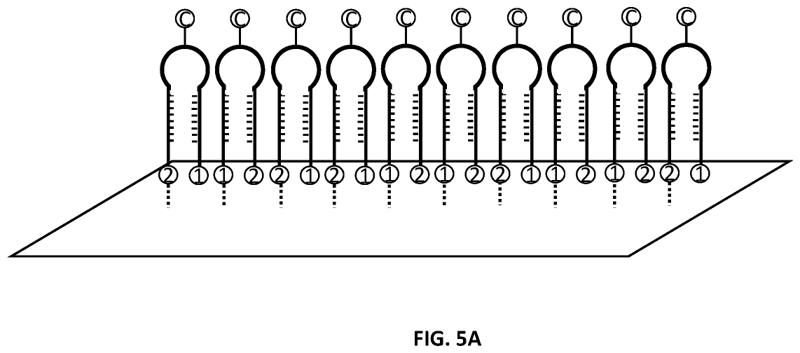

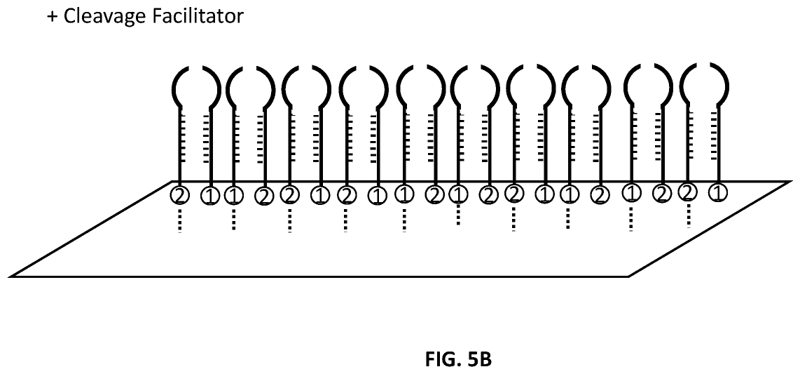

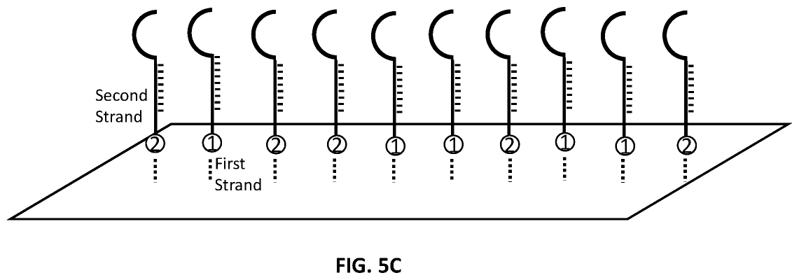

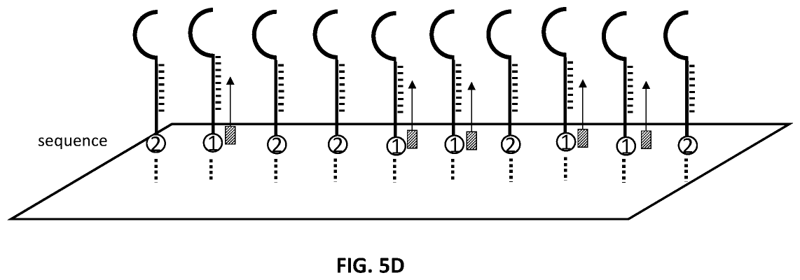

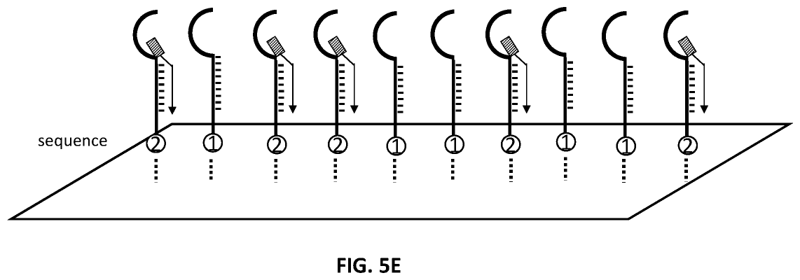

FIGS. 5A-5E illustrates another embodiment of two-strand complex sequencing for providing Duplex Sequencing information on a sequencing surface (e.g., flow cell). In the embodiment illustrated in FIGS. 5A-5E, sequence reads from both the first and second strands of the original adapter-nucleic acid complexes can be generated without a second bridge amplification step. As discussed above, each two-stranded complex can be independently bridge amplified on a surface to generate a clonal cluster comprising multiple of copies of the two-strand complex having both a first strand and a complementary second strand with an intervening hairpin linker domain with a cleavable site (FIG. 5A). The copies can be in both the forward orientation and the reverse orientation as discussed above.

As shown in FIG. 5B, and in one embodiment, the two-strand complexes may be cleaved at the cleavage site C (e.g., via a cleavage facilitator as discussed further herein). Following cleavage at site C, the non-bound strand is removed. Referring to FIG. 5C, the remaining molecules bound to the surface of the flow cell include (a) first strand sequences in a reverse orientation (e.g., adjacent to primer site “1”), and (b) second strand sequences in the forward orientation (e.g., adjacent to primer site “2).

In a next step, a first sequencing reaction using a primer specific to the reverse orientation is used to obtain sequencing information for the first strand (FIG. 5D). The primer(s) used in the first sequencing reaction can be washed away. In a next step, a second sequencing reaction using a primer specific to the foward orientation is used to obtain sequencing information for the second strand (FIG. 5E). The embodiment illustrated in FIGS. 5D and 5E show sequencing the first and second strands consecutively. It will be understood that, in another embodiment, the first and second strands can be sequenced simultaneously (e.g., in the same sequencing reaction) using, for example, multiple color chemistry (e.g., 4 color chemistry) followed by deconvolution of the sequencing/color frequency signals to determine the origin of a particular sequencer base call or signal.

Once sequencing reads from the first strand and the second strands are generated, the first strand sequencing read can be compared to the second strand sequencing read for providing Duplex error correction. The embodiments described herein overcome some of the challenges associated with conversion efficiency described above in that sequencing information from each clonal cluster provides both the first strand sequencing read and the second strand sequencing read.

II. Embodiments of Method and Reagents for Cleaving Hairpin Adapters.

Conventionally, sequencing reactions of hairpin linked adapter-nucleic acid complexes may be difficult, as a polymerase must displace hybridized regions of self-complementarity. For example, due to the close proximity of the self-complementary portions of the adapter-nucleic acid complexes, and because the melting temperature (Tm) of the complementary portions of the first and second strands is high, polymerase-based sequencing of such structures remain a barrier to providing Duplex Sequencing data of physically linked strands.

As discussed above, aspects of the present technology incorporate use of hairpin adapters having a cleavable site or motif such that first and second strand nucleic acid sequences can be separated from each other during a sequencing reaction.

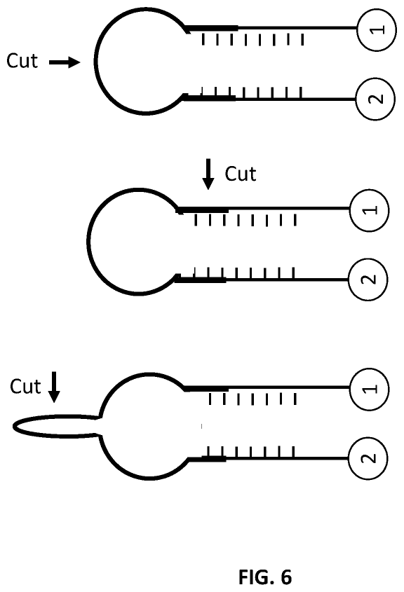

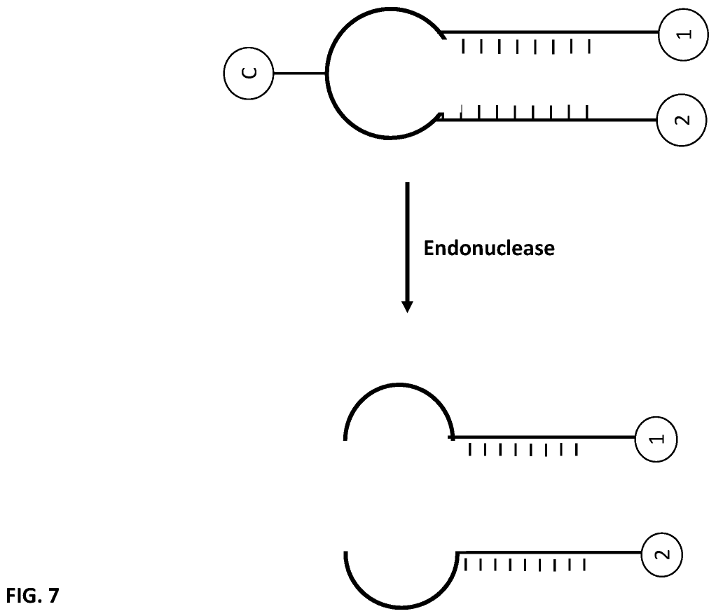

In certain embodiments, and as illustrated in FIG. 6, the hairpin adapter can comprise (e.g., in a single-stranded portion or in a double-stranded portion, a cleavage motif that allows for the subsequent cleavage of the hairpin DNA molecule by an enzyme (e.g., an endonuclease) or other cleavage facilitator (chemical or non-enzymatic process). With reference to FIG. 7, and in one embodiment, a single-stranded (e.g., linker region) of the hairpin adapter can be cleaved using an endonuclease (e.g., a restriction site endonuclease, a target endonuclease, etc.). For example, FIG. 7 illustrates a single-stranded cleavage site (e.g., nucleic acid sequence) that is digestible by an endonuclease (e.g., a restriction enzyme). With reference to FIGS. 3A-5E and 7, and after bridge amplification of the two-strand complexes, an enzyme can be introduced (e.g., flow through the flow cell) to cleave at the cleavage site. In some embodiments, inefficient cleavage is desired (e.g., some uncleaved two-strand complexes remaining is desirable to seed the second round of bridge amplification). In some embodiments, an enzymatic reaction can be time or concentration controlled such that a portion of two-stranded complexes with be cleaved and a portion will remain uncleaved. For example, a limited amount of restriction enzyme could be flowed across the functionalized surface in order to cut the majority, but not all, of the hairpin DNA molecules. In another embodiment, a restriction enzyme could be flowed across the surface for a limited amount of time in order to cut the majority, but not all, of the hairpin DNA molecules. In another embodiment, a mixture of enzymes, in which the majority are catalytically active, and a small amount are catalytically inactive, could be flowed across the functionalized surface in order to cut the majority, but not all, of the hairpin DNA molecules.

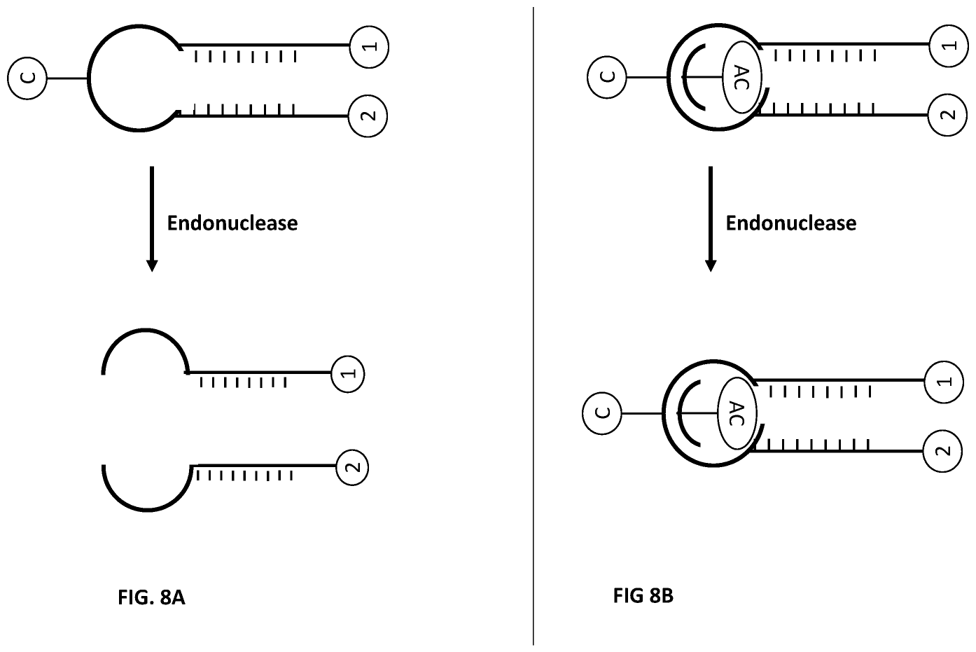

FIGS. 8A and 8B illustrate another embodiment for providing a cleavage site in a linker domain of a hairpin adapter in a manner that allows for inefficient cleavage of two-stranded complexes in a clonal cluster. In this example, and prior to introduction of an endonuclease, the method can provide for introduction of an oligonucleotide at least partially complementary to the linker domain of the hairpin adapter. As shown in FIG. 8B, hybridization of the introduced oligonucleotide can prevent cleavage (e.g., provide an anti-cleavage motif “AC”) by the endonuclease. Two-stranded complexes that do not have a hybridized oligo (FIG. 8A) remain susceptible to cleavage by the endonuclease. The concentration of oligonucleotide provided to the sequencing flow cell, prior to enzymatic cleavage (or concurrent with endonuclease introduction), can be scalable to retain the desirable number of uncleaved complexes within each clonal cluster on the flow cell. For example, a small amount of an oligonucleotide sequence containing an anti-cleavage motif can be flowed across the functionalized surface, resulting in the hybridization of the oligonucleotide sequence to a subset (e.g., a limited amount) of the hairpin DNA molecules in each clonal cluster (FIG. 8B). The majority of the hairpin DNA molecules (containing a cleavage motif within the hairpin) will not be hybridized to the oligonucleotide sequence containing the anti-cleavage motif. As such, the majority of the hairpin DNA molecules (that are not hybridized to the oligonucleotide sequence containing the anti-cleavage motif) can be cleaved at the single-stranded cleavage motif within the hairpin adapter. The hairpin DNA molecules that are hybridized to the oligonucleotide sequence containing the anti-cleavage motif remain uncut by the enzyme.

In one embodiment, the cleavage motif within the hairpin adapter can be methylated, and the anti-cleavage motif within the oligonucleotide sequence can be non-methylated. An enzyme that only cuts methylated DNA can then be flowed across the functionalized surface. In another embodiment, the cleavage motif within the hairpin adapter can be non-methylated, and the anti-cleavage motif within the oligonucleotide sequence can be methylated. An enzyme that only cuts non-methylated DNA can then be flowed across the functionalized surface. In another embodiment, the anti-cleavage motif within the oligonucleotide sequence can be a side chain that prevents the hairpin DNA molecule from being cleaved. In another embodiment, the anti-cleavage motif within the oligonucleotide sequence can be a bulky adduct that prevents the hairpin DNA molecule from being cleaved. In another embodiment, an anti-cleavage motif within the oligonucleotides sequence can be one or more mismatches that prevent the enzyme from cutting the hairpin DNA molecule. In another embodiment, the anti-cleavage motif can be an abasic site that prevents cleavage. In another embodiment, the anti-cleavage motif can be a nucleotide analogue that prevents cleavage. In another embodiment, the anti-cleavage motif can be a peptide-nucleic acid bond that prevents cleavage.

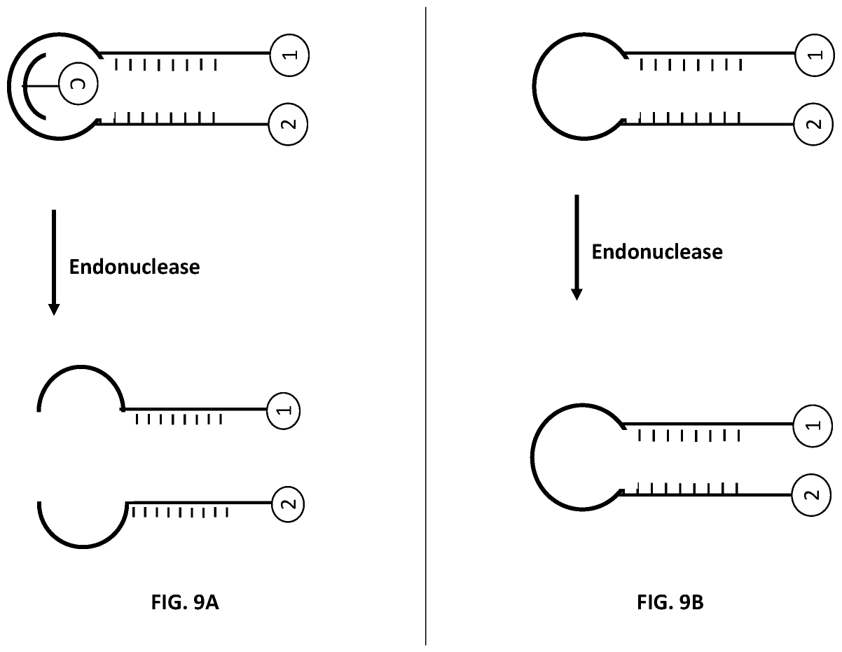

In another embodiment shown in FIGS. 9A-9B, an oligonucleotide comprising an at least partially complementary sequence to the linker domain of the hairpin adapter can be provided to hybridize with the linker domain and form a cleavage site/motif. For example, an endonuclease that recognizes a double-strand cutting site, can be used to cut linker regions comprising the double-stranded region provided by the hybridized oligonucleotide (FIG. 9A). For example, an oligonucleotide can be flowed across the functionalized surface, resulting in the hybridization of the oligonucleotide sequence to the linker region of the hairpin adapter and thereby providing a double-stranded cleavage motif in a portion of the hairpin DNA molecules (FIG. 9A). In one embodiment, a limited amount of the oligonucleotide can be flowed across the functionalized surface in order for hybridization between the oligonucleotide sequence and the hairpin DNA molecule to occur for some, but not all, of the hairpin DNA molecules. In another embodiment, the oligonucleotide can be flowed across the functionalized surface for a limited amount of time in order for hybridization between the oligonucleotide sequence and the hairpin DNA molecule to occur for some, but not all, of the hairpin DNA molecules. The hairpin DNA molecules that are hybridized to the oligonucleotide sequence thereby providing a cleavage motif are cleaved following the flow of an endonuclease across the functionalized surface. The hairpin DNA molecules not hybridized to the oligonucleotide sequence containing a cleavage motif remain uncleaved.

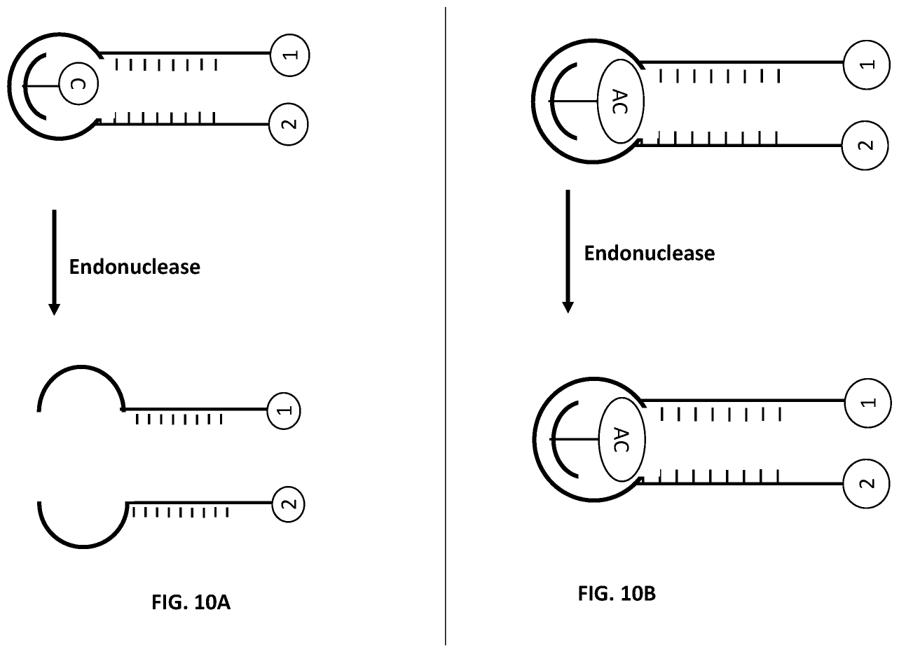

In yet another embodiment, illustrated in FIGS. 10A-10B, a pool of oligonucleotides comprising at least partially complementary sequences to the linker domain of the hairpin adapter can be provided to hybridize with the linker domain. The pool of oligonucleotides can include a subset of oligonucleotides, that once hybridized, provide a cleavage site/motif (e.g., for a suitable endonuclease) (FIG. 10A). The pool of oligonucleotides can also include a subset of oligonucleotides, that once hybridized, provide an ani-cleavage motif (and/or prevent cleavage by, for example, disrupting site recognition by the endonuclease) (FIG. 10B). In one example, the pool of oligonucleotides can be flowed across the functionalized surface. The hairpin DNA molecules that are hybridized to the oligonucleotide sequence containing a cleavage motif are cleaved, and the hairpin DNA molecules hybridized to the oligonucleotide sequence containing the anti-cleavage motif remain un-cleaved. In one embodiment, the one subset of the oligonucleotides can be methylated, and the second subset of oligonucleotides can be non-methylated. In one embodiment, an enzyme that only cuts methylated DNA can then be flowed across the functionalized surface. In another embodiment, an enzyme that only cleaves unmethylated DNA can be flowed across the functionalized surface. In another embodiment, the oligonucleotide providing the anti-cleavage motif can comprise a side chain that prevents the hairpin DNA molecule from being cleaved. In another embodiment, the anti-cleavage motif within the oligonucleotide sequence can be a bulky adduct that prevents the hairpin DNA molecule from being cleaved. In another embodiment, the anti-cleavage motif within the oligonucleotides sequence can be one or more mismatches that prevent the enzyme from cutting the hairpin DNA molecule. In another embodiment, the anti-cleavage motif can be an abasic site that prevents cleavage. In another embodiment, the anti-cleavage motif can be a nucleotide analogue that prevents cleavage. In another embodiment, the anti-cleavage motif can be a peptide-nucleic acid bond that prevents cleavage. Those of ordinary skill in the art will recognize other biochemical means for providing a subset of oligonucleotides that will prevent or facilitate cleavage by a selected endonuclease or other enzyme.

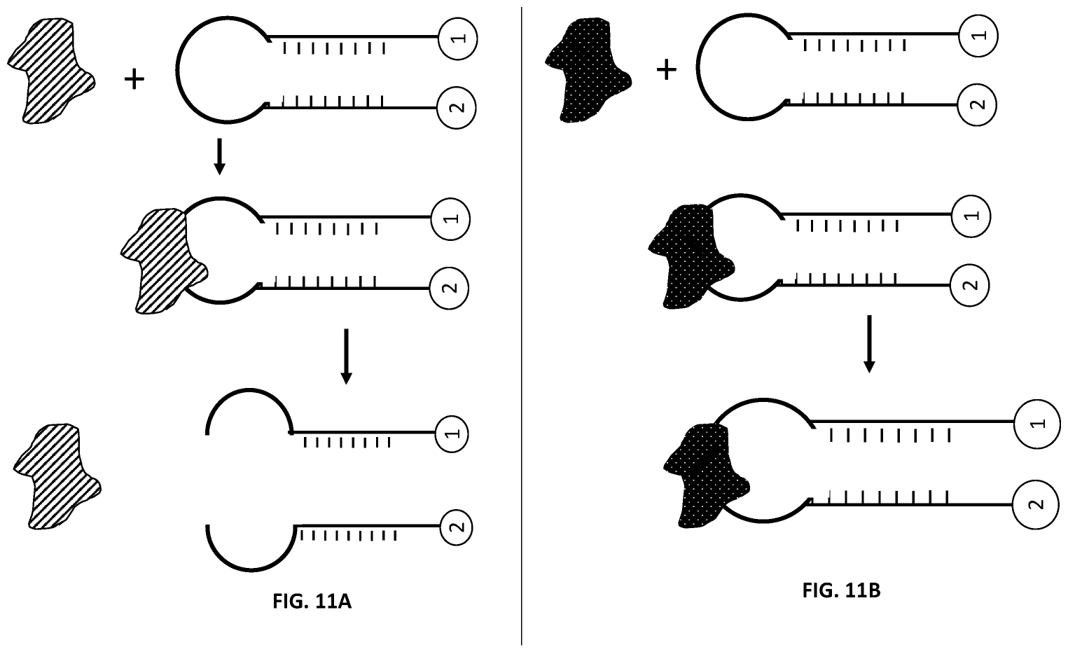

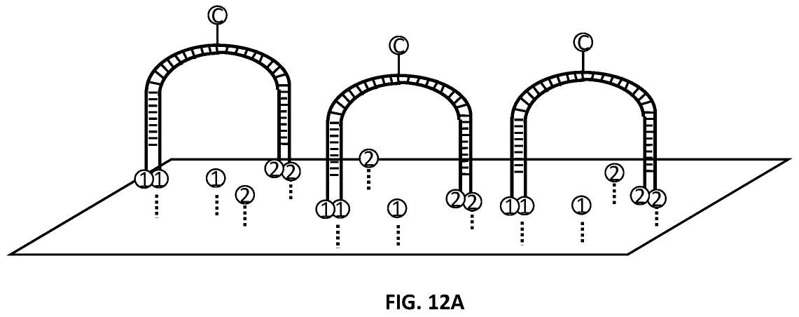

In yet a further embodiment, and as illustrated in FIGS. 11A and 11B, inefficient cleavage of a portion of the clonal copies of the two-stranded nucleic acid complexes can be accomplished by use of mixed pool of endonucleases having a portion of catalytically active enzyme (striped; FIG. 11A) and a portion of catalytically inactive enzyme (black with dots; FIG. 11B).

In some embodiments, an endonuclease is or comprises a targeted endonuclease. In some embodiments, a targeted endonuclease is or comprises at least one of a restriction endonuclease (i.e., restriction enzyme) that cleaves DNA at or near recognition sites (e.g., EcoRI, BamHI, XbaI, HindIII, AluI, AvaII, BsaJI, BstNI, DsaV, Fnu4HI, HaeIII, MaeIII, N1aIV, NSiI, MspJI, FspEI, Nael, Bsu36I, NotI, HinF1, Sau3AI, PvuII, SmaI, HgaI, AluI, EcoRV, etc.). Listings of several restriction endonucleases are available both in printed and computer readable forms, and are provided by many commercial suppliers (e.g., New England Biolabs, Ipswich, Mass.). It will be appreciated by one of ordinary skill in the art that any restriction endonuclease may be used in accordance with various embodiments of the present technology. In other embodiments, a targeted endonuclease is or comprises at least one of a ribonucleoprotein complex, such as, for example, a CRISPR-associated (Cas) enzyme/guideRNA complex (e.g., Cas9 or Cpf1) or a Cas9-like enzyme. In other embodiments, a targeted endonuclease is or comprises a homing endonuclease, a zinc-fingered nuclease, a TALEN, and/or a meganuclease (e.g., megaTAL nuclease, etc.), an argonaute nuclease or a combination thereof. In some embodiments, a targeted endonuclease comprises Cas9 or CPF1 or a derivative thereof. In another embodiment, a nuclease can cut at a forked nucleic region (e.g., FEN1). In some embodiments, more than one targeted endonuclease may be used (e.g., 2, 3, 4, 5, 6, 7, 8, 9, 10 or more).

In some embodiments, a cut site is or comprises a user-directed recognition sequence for a targeted endonuclease (e.g., a CRISPR or CRISPR-like endonuclease) or other tunable endonuclease. In some embodiments, cutting nucleic acid material may comprise at least one of enzymatic digestion, enzymatic cleavage, enzymatic cleavage of one strand, enzymatic cleavage of both strands, incorporation of a modified nucleic acid followed by enzymatic treatment that leads to cleavage or one or both strands, incorporation of a replication blocking nucleotide, incorporation of a chain terminator, incorporation of a photocleavable linker, incorporation of a uracil, incorporation of a ribose base, incorporation of an 8-oxo-guanine adduct, use of a restriction endonuclease, use of a ribonucleoprotein endonuclease (e.g., a Cas-enzyme, such as Cas9 or CPF1), or other programmable endonuclease (e.g., a homing endonuclease, a zinc-fingered nuclease, a TALEN, a meganuclease (e.g., megaTAL nuclease), an argonaute nuclease, etc.), and any combination thereof.

Targeted endonucleases (e.g., a CRISPR-associated ribonucleoprotein complex, such as Cas9 or Cpf1 , a homing nuclease, a zinc-fingered nuclease, a TALEN, a megaTAL nuclease, an argonaute nuclease, and/or derivatives thereof) can be used to selectively cut targeted portions of nucleic acid material. In some embodiments, a targeted endonuclease can be modified, such as having an amino acid substitution for provided, for example, enhanced thermostability, salt tolerance and/or pH tolerance or enhanced specificity or alternate PAM site recognition or higher affinity for binding. In other embodiments, a targeted endonuclease may be biotinylated, fused with streptavidin and/or incorporate other affinity-based (e.g., bait/prey) technology. In certain embodiments, a targeted endonuclease may have an altered recognition site specificity (e.g., SpCas9 variant having altered PAM site specificity). In other embodiments, a targeted endonuclease may be catalytically inactive so that cleavage does not occur once bound to targeted portions of nucleic acid material. In some embodiments, a targeted endonuclease is modified to cleave a single strand of a targeted portion of nucleic acid material (e.g., a nickase variant) thereby generating a nick in the nucleic acid material. CRISPR-based targeted endonucleases are further discussed herein to provide a further detailed non-limiting example of use of a targeted endonuclease. We note that the nomenclature around such targeted nucleases remains in flux. For purposes herein, we use the term “CRISPR-based” to generally mean endonucleases comprising a nucleic acid sequence, the sequence of which can be modified to redefine a nucleic acid sequence to be cleaved. Cas9 and CPF1 are examples of such targeted endonucleases currently in use, but many more appear to exist different places in the natural world and the availability of different varieties of such targeted and easily tunable nucleases is expected to grow rapidly in the coming years. For example, Cas12a, Cas13, CasX and others are contemplated for use in various embodiments. Similarly, multiple engineered variants of these enzymes to enhance or modify their properties are becoming available. Herein, we explicitly contemplate use of substantially functionally similar targeted endonucleases not explicitly described herein or not yet discovered, to achieve a similar purpose to disclosures described within.

It is specifically contemplated that any of a variety of restriction endonucleases (i.e., enzymes) may be used. Generally, restriction enzymes are typically produced by certain bacteria/other prokaryotes and cleave at, near or between particular sequences in a given segment of DNA.

It will be apparent to one of skill in the art that a restriction enzyme is chosen to cut at a particular site or, alternatively, at a site that is generated in order to create a restriction site for cutting. In some embodiments, a restriction enzyme is a synthetic enzyme. In some embodiments, a restriction enzyme is not a synthetic enzyme. In some embodiments, a restriction enzyme as used herein has been modified to introduce one or more changes within the genome of the enzyme itself. In some embodiments, restriction enzymes produce double-stranded cuts between defined sequences within a given portion of DNA.

While any restriction enzyme may be used in accordance with some embodiments (e.g., type I, type II, type III, and/or type IV), the following represents a non-limiting list of restriction enzymes that may be used: AluI, ApoI, AspHI, BamHI, BfaI, BsaI, CfrI, DdeI, DpnI, DraI, EcoRI, EcoRII, EcoRV, HaeII, HaeIII, HgaI, HindII, HindIII, HinFI, HPYCH4III, KpnI, MamI, MNL1, MseI, MstI, MstII, NcoI, NdeI, NotI, PacI, PstI, PvuI, PvuII, RcaI, RsaI, SacI, SacII, SalI, Sau3AI, ScaI, SmaI, SpeI, SphI, StuI, TaqI, XbaI, XhoI, XhoII, XmaI, XmaII, and any combination thereof. An extensive, but non-exhaustive list of suitable restriction enzymes can be found in publicly-available catalogues and on the internet (e.g., available at New England Biolabs, Ipswich, Mass., U.S.A.). It is understood by one experienced in the art that a variety of enzymes, ribozymes or other nucleic acid modifying enzymes that can, alone or in combination, be used to target phosphodiester backbone cleavage of a nucleic acid molecule that can achieve the same purpose may not be included or yet discovered on the above list. A variety of nucleic acid modifying enzymes can recognize base modifications (e.g. CpG methylation) which can be used to target further modification of the adjacent nucleic acid sequence (e.g. to generate an abasic site) that can be cleaved (e.g. by an enzyme with lyase activity). As such, substantial sequence specificity of cleavage can be achieved based on recognition of DNA or RNA modifications and this can be used alone or in combination with targeted endonucleases to achieve targeted nucleic acid fragmentation. Other embodiments of cleavage facilitators can comprise non-enzymatic facilitators. For example, pH changes or hydrolysis can be used to cleave at the cleavage site. Photocleavage methods are also an approach to break this backbone. For example, incorporation of a modified nucleotide in the hairpin adapter sequence or hybridization of a complementary or partially complementary oligonucleotide having a photosensitive moiety can create a recognition site for other chemical or enzymatic processes that would cleave (e.g., upon exposure to light) the opposite strand.

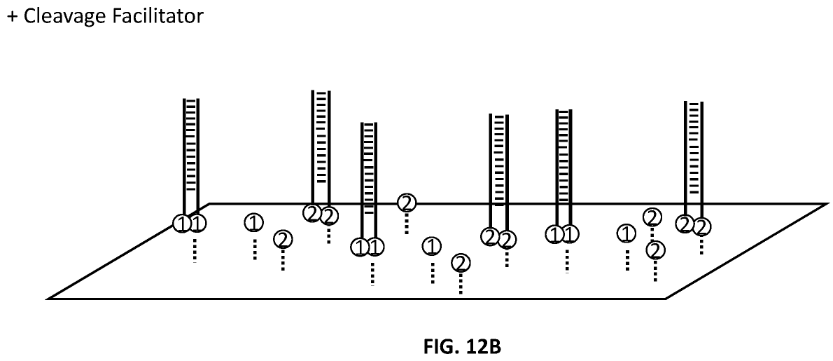

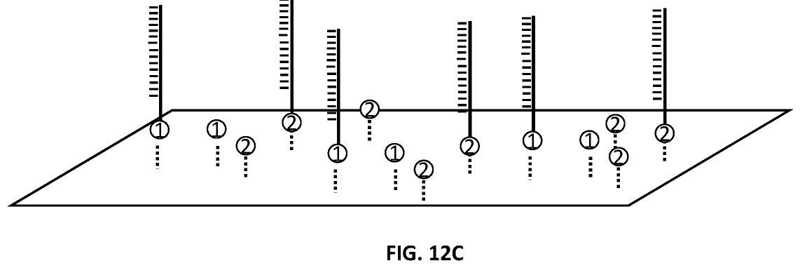

In some embodiments, such as those described above, the cleavage site C is provided when the physically-linked adapter-molecule complexes are in a self-hybridized configuration on the surface (e.g., FIGS. 6, 7, 8A, 9A, 10A, and 11A, for example). In yet a further embodiment, and as illustrated in FIGS. 12A-C, the cleavage cite C is available for cleavage by a cleavage facilitator when the physically-linked nucleic acid complexes or in a double-stranded bridge amplified configuration. For example, the cleavage site C is a double-stranded motif provided by the double-stranded configuration following double-strand formation across the “bridge” on the surface, but before denaturation (FIG. 12A). Once cleaved, the first strand sequence amplicons will be separated from the second strand amplicons while still bound to the surface (FIG. 12B). Following denaturation and removal of the unbound amplicons (FIG. 12C), single-stranded amplicons of both the first strand and the second strand remain bound and available to sequence. In one embodiment, sequencing of the first and second strand amplicons can proceed with sequencing reactions such as those described with respect to FIGS. 5D and 5E.

Adapters

As described above, adapter molecules can be or comprise “Y”-shaped, “U”-shaped, “hairpin” shaped, have a bubble (e.g., a portion of sequence that is non-complimentary), or other features. A “U”-shaped or “hairpin” shaped adapter can refer to an adapter with a linker domain that links or connects a first strand of a target double-stranded nucleic acid molecule to a second strand of the same molecule. Certain hairpin adapters, for example, can be cleavable hairpin adapters and/or may comprise modified or non-standard nucleotides, restriction sites, or other features for manipulation of structure or function in vitro.

Adapter molecules may ligate to a variety of nucleic acid material having a terminal end. For example, adapter molecules can be suited to ligate to a T-overhang, an A-overhang, a CG-overhang, a multiple nucleotide overhang (also referred to herein as a “sticky end” or “sticky overhang”) or single-stranded overhang region with known nucleotide length (e.g., 1, 2, 3, 4, 5, 6, 7, 8, 9, 10, 11, 12, 13, 14, 15, 16, 17, 18, 19, 20 or more nucleotides), a dehydroxylated base, a blunt end of a nucleic acid material and the end of a molecule were the 5′ of the target is dephosphorylated or otherwise blocked from traditional ligation. In other embodiments the adapter molecule can contain a dephosphorylated or otherwise ligation-preventing modification on the 5′ strand at the ligation site. In the latter two embodiments such strategies may be useful for preventing dimerization of library fragments or adapter molecules.

The ligation domain of an adapter can be cleaved with an endonuclease (e.g., restriction endonuclease, targeted endonuclease, etc.) enzyme to leave a 3′ “T” overhang which is compatible for ligation with a 3′ “A” overhang in a prepared library fragment. In certain embodiments the resulting ligation domain is a single base pair thymine (T) overhang on the 3′ end of the extended extension strand, but in other embodiments, it can be a blunt end, or a different type or 3′ or 5′ overhang “sticky” end. In this particular example “CUT” implies use of a sequence-specific endonuclease, such as a restriction enzyme, to cleave in a way that inherently creates the ligateable end. In other embodiments, after cleavage, further enzymatic or chemical processing, such as with a terminal transferase, can create the ligateable end.

Referring back to FIG. 2A, the ligateable end is shown as a T-overhang, however, it will be apparent to one of skill in the art that the ligateable end can be any of a variety of forms, for example, a blunt end, an A-3′ overhang, a “sticky” end comprising a one nucleotide 3′ overhang, a two nucleotide 3′ overhang, a three nucleotide 3′ overhang, a 4, 5, 6, 7, 8, 9, 10, 11, 12, 13, 14, 15, 16, 17, 18, 19, 20 or more nucleotide 3′ overhang, a one nucleotide 5′ overhang, a two nucleotide 5′ overhang, a three nucleotide 5′ overhang, a 4, 5, 6, 7, 8, 9, 10, 11, 12, 13, 14, 15, 16, 17, 18, 19, 20 or more nucleotide 5′ overhang, among others (e.g., FIG. 2B). The 5′ base of the ligation site can be phosphorylated and the 3′ base can have a hydroxyl group, or either can be, alone or in combination, dephosphorylated or dehydrated or further chemically modified to either facilitate enhanced ligation or one strand to prevent ligation of one strand, optionally, until a later time point.

In some embodiments, adapter molecules can comprise a capture moiety suitable for isolating a desired target nucleic acid molecule ligated thereto.

An adapter sequence can mean a single-strand sequence, a double-strand sequence, a complimentary sequence, a non-complimentary sequence, a partial complimentary sequence, an asymmetric sequence, a primer binding sequence, a flow-cell sequence, a ligation sequence or other sequence provided by an adapter molecule. In particular embodiments, an adapter sequence can mean a sequence used for amplification by way of compliment to an oligonucleotide.

In some embodiments, provided methods and compositions include at least one adapter sequence (e.g., two adapter sequences, one on each of the 5′ and 3′ ends of a nucleic acid material). In some embodiments, provided methods and compositions may comprise 2 or more adapter sequences (e.g., 3, 4, 5, 6, 7, 8, 9, 10 or more). In some embodiments, at least two of the adapter sequences differ from one another (e.g., by sequence). In some embodiments, each adapter sequence differs from each other adapter sequence (e.g., by sequence). In some embodiments, at least one adapter sequence is at least partially non-complementary to at least a portion of at least one other adapter sequence (e.g., is non-complementary by at least one nucleotide).

In some embodiments, an adapter sequence comprises at least one non-standard nucleotide. In some embodiments, a non-standard nucleotide is selected from an abasic site, a uracil, tetrahydrofuran, 8-oxo-7,8-dihydro-2′deoxyadenosine (8-oxo-A), 8-oxo-7,8-dihydro-2′-deoxyguanosine (8-oxo-G), deoxyinosine, 5′nitroindole, 5-Hydroxymethyl-2′-deoxycytidine, iso-cytosine, 5-methyl-isocytosine, or isoguanosine, a methylated nucleotide, an RNA nucleotide, a ribose nucleotide, an 8-oxo-guanine, a photocleavable linker, a biotinylated nucleotide, a desthiobiotin nucleotide, a thiol modified nucleotide, an acrydite modified nucleotide an iso-dC, an iso dG, a 2′-O-methyl nucleotide, an inosine nucleotide Locked Nucleic Acid, a peptide nucleic acid, a 5 methyl dC, a 5-bromo deoxyuridine, a 2,6-Diaminopurine, 2-Aminopurine nucleotide, an abasic nucleotide, a 5-Nitroindole nucleotide, an adenylated nucleotide, an azide nucleotide, a digoxigenin nucleotide, an I-linker, an 5′ Hexynyl modified nucleotide, an 5-Octadiynyl dU, photocleavable spacer, a non-photocleavable spacer, a click chemistry compatible modified nucleotide, and any combination thereof.

In some embodiments, an adapter sequence comprises a moiety having a magnetic property (i.e., a magnetic moiety). In some embodiments this magnetic property is paramagnetic. In some embodiments where an adapter sequence comprises a magnetic moiety (e.g., a nucleic acid material ligated to an adapter sequence comprising a magnetic moiety), when a magnetic field is applied, an adapter sequence comprising a magnetic moiety is substantially separated from adapter sequences that do not comprise a magnetic moiety (e.g., a nucleic acid material ligated to an adapter sequence that does not comprise a magnetic moiety).

In some embodiments, at least one adapter sequence is located 5′ to a SMI. In some embodiments, at least one adapter sequence is located 3′ to a SMI.

In some embodiments, an adapter sequence may comprise one or more linker domains. In some embodiments, a linker domain may be comprised of nucleotides. In some embodiments, a linker domain may include at least one modified nucleotide or non-nucleotide molecules (for example, as described elsewhere in this disclosure). In some embodiments, a linker domain may be or comprise a loop.

In some embodiments, an adapter sequence on either or both ends of each strand of a double-stranded nucleic acid material may further include one or more elements that provide a SDE. In some embodiments, a SDE may be or comprise asymmetric primer sites comprised within the adapter sequences.

In some embodiments, an adapter sequence may be or comprise at least one SDE and at least one ligation domain (i.e., a domain amendable to the activity of at least one ligase, for example, a domain suitable to ligating to a nucleic acid material through the activity of a ligase). In some embodiments, from 5′ to 3′, an adapter sequence may be or comprise a primer binding site, a SDE, and a ligation domain.

Various methods for synthesizing Duplex Sequencing adapters have been previously described in, e.g., U.S. Pat. No. 9,752,188, International Patent Publication No. WO2017/100441, and International Patent Application No. PCT/US18/59908 (filed Nov. 8, 2018), all of which are incorporated by reference herein in their entireties.

Various methods for synthesizing Duplex Sequencing adapters have been previously described (e.g., U.S. Pat. No. 9,752,188 and U.S. Patent No. PCT/US19/17908, incorporated by reference herein). For example, and in one embodiment, one oligonucleotide can be hybridized to another oligonucleotide containing a degenerate or semidegenerate nucleotide sequence on a region of non-complementarity. The hybridized oligonucleotides may then be chemically linked, or may be two portions of a continuous oligonucleotide that, when hybridized, forms a “loop” or a “U” shape (a hairpin adapter). An enzyme capable of polymerizing nucleotides can then be used to copy a single-stranded degenerate or semidegenerate region such that a complement is synthesized. A now complementary double-stranded degenerate or semi-degenerate sequence is thus produced, which may serve as the at least one SMI element during Duplex Sequencing. The ligation site on the adapter molecule may be modified from this extension product by enzymatic or chemical manipulation (for example, by restriction digestion, terminal transferase activity of a polymerase, or other enzyme or any other method known in the art).

Primers

In some embodiments, one or more PCR primers that have at least one of the following properties: 1) high target specificity; 2) capable of being multiplexed; and 3) exhibit robust and minimally biased amplification are contemplated for use in various embodiments in accordance with aspects of the present technology. A number of prior studies and commercial products have designed primer mixtures satisfying certain of these criteria for conventional PCR-CE. However, it has been noted that these primer mixtures are not always optimal for use with MPS. Indeed, developing highly multiplexed primer mixtures can be a challenging and time-consuming process. Conveniently, both Illumina and Promega have recently developed multiplex compatible primer mixtures for the Illumina platform that show robust and efficient amplification of a variety of standard and non-standard STR and SNP loci. Because these kits use PCR to amplify their target regions prior to sequencing, the 5′-end of each read in paired-end sequencing data corresponds to the 5′-end of the PCR primers used to amplify the DNA. In some embodiments, provided methods and compositions include primers designed to ensure uniform amplification, which may entail varying reaction concentrations, melting temperatures, and minimizing secondary structure and intra/inter-primer interactions. Many techniques have been described for highly multiplexed primer optimization for MPS applications. In particular, these techniques are often known as ampliseq methods, as well described in the art.

Amplification

Provided methods and compositions, in various embodiments, make use of, or are of use in, at least one amplification step wherein a nucleic acid material (or portion thereof, for example, a specific target region or locus) is amplified to form an amplified nucleic acid material (e.g., some number of amplicon products).

In some embodiments, amplifying a nucleic acid material includes a step of amplifying nucleic acid material derived from each of a first and second nucleic acid strand from an original double-stranded nucleic acid material using at least one single-stranded oligonucleotide at least partially complementary to a sequence present in a first adapter sequence. An amplification step further includes employing a second single-stranded oligonucleotide to amplify each strand of interest, and such second single-stranded oligonucleotide can be (a) at least partially complementary to a target sequence of interest, or (b) at least partially complementary to a sequence present in a second adapter sequence such that the at least one single-stranded oligonucleotide and a second single-stranded oligonucleotide are oriented in a manner to effectively amplify the nucleic acid material.

In some embodiments, amplifying nucleic acid material in a sample can include amplifying nucleic acid material in “tubes” (e.g., PCR tubes), in emulsion droplets, microchambers, and other examples described above or other known vessels. In some embodiments, amplifying nucleic acid material may comprise amplifying nucleic acid material in two or more (e.g., 3, 4, 5, 6, 7, 8, 9, 10, 20, 30, 40, 50 or more samples) physically separated samples (e.g., tubes, droplets, chambers, vessels, etc.).

While any application-appropriate amplification reaction is contemplated as compatible with some embodiments, by way of specific example, in some embodiments, an amplification step may be or comprise a polymerase chain reaction (PCR), rolling circle amplification (RCA), multiple displacement amplification (MDA), isothermal amplification, polony amplification within an emulsion, bridge amplification on a surface, the surface of a bead or within a hydrogel, and any combination thereof.

In some embodiments, amplification on a surface, such as bridge amplification on a surface of a flow cell, includes generating clusters or multiple of copies of bound nucleic acid template. In a particular embodiment, linked first and second strand nucleic acid templates can bridge amplify on the surface of a flow cell, for example, to generate a plurality of clonal clusters, wherein each clonal cluster comprises nucleic acid template copies derived from both the original first and second strands of the original double-stranded nucleic acid molecule. Some of the clonal copies in a cluster will be in the forward orientation, while the rest will be in the reverse origination. A sequencing reaction can proceed when either the copies in the forward orientation or the copies in the reverse orientation is first cleaved and removed.

In some embodiments, amplifying a nucleic acid material includes use of single-stranded oligonucleotides at least partially complementary to regions of the adapter sequences on the 5′ and 3′ ends of each strand of the nucleic acid material. In some embodiments, amplifying a nucleic acid material includes use of at least one single-stranded oligonucleotide at least partially complementary to a target region or a target sequence of interest (e.g., a genomic sequence, a mitochondrial sequence, a plasmid sequence, a synthetically produced target nucleic acid, etc.) and a single-stranded oligonucleotide at least partially complementary to a region of the adapter sequence (e.g., a primer site).

In general, robust amplification, for example PCR amplification, can be highly dependent on the reaction conditions. Multiplex PCR, for example, can be sensitive to buffer composition, monovalent or divalent cation concentration, detergent concentration, crowding agent (i.e. PEG, glycerol, etc.) concentration, primer concentrations, primer Tms, primer designs, primer GC content, primer modified nucleotide properties, and cycling conditions (i.e. temperature and extension times and rate of temperature changes). Optimization of buffer conditions can be a difficult and time-consuming process. In some embodiments, an amplification reaction may use at least one of a buffer, primer pool concentration, and PCR conditions in accordance with a previously known amplification protocol. In some embodiments, a new amplification protocol may be created, and/or an amplification reaction optimization may be used. By way of specific example, in some embodiments, a PCR optimization kit may be used, such as a PCR Optimization Kit from Promega®, which contains a number of pre-formulated buffers that are partially optimized for a variety of PCR applications, such as multiplex, real-time, GC-rich, and inhibitor-resistant amplifications. These pre-formulated buffers can be rapidly supplemented with different Mg2+ and primer concentrations, as well as primer pool ratios. In addition, in some embodiments, a variety of cycling conditions (e.g., thermal cycling) may be assessed and/or used. In assessing whether or not a particular embodiment is appropriate for a particular desired application, one or more of specificity, allele coverage ratio for heterozygous loci, interlocus balance, and depth, among other aspects may be assessed. Measurements of amplification success may include DNA sequencing of the products, evaluation of products by gel or capillary electrophoresis or HPLC or other size separation methods followed by fragment visualization, melt curve analysis using double-stranded nucleic acid binding dyes or fluorescent probes, mass spectrometry or other methods known in the art.

In some embodiments, at least one amplifying step includes at least one primer that is or comprises at least one non-standard nucleotide. In some embodiments, a non-standard nucleotide is selected from a uracil, a methylated nucleotide, an RNA nucleotide, a ribose nucleotide, an 8-oxo-guanine, a biotinylated nucleotide, a locked nucleic acid, a peptide nucleic acid, a high-Tm nucleic acid variant, an allele discriminating nucleic acid variant, any other nucleotide or linker variant described elsewhere herein and any combination thereof.

Nucleic Acid Material

Types

In accordance with various embodiments, any of a variety of nucleic acid material may be used. In some embodiments, nucleic acid material may comprise at least one modification to a polynucleotide within the canonical sugar-phosphate backbone. In some embodiments, nucleic acid material may comprise at least one modification within any base in the nucleic acid material. For example, by way of non-limiting example, in some embodiments, the nucleic acid material is or comprises at least one of double-stranded DNA, single-stranded DNA, double-stranded RNA, single-stranded RNA, peptide nucleic acids (PNAs), locked nucleic acids (LNAs).

Sources

It is contemplated that nucleic acid material may come from any of a variety of sources. For example, in some embodiments, nucleic acid material is provided from a sample from at least one subject (e.g., a human or animal subject) or other biological source. In some embodiments, a nucleic acid material is provided from a banked/stored sample. In some embodiments, a sample is or comprises at least one of blood, serum, sweat, saliva, cerebrospinal fluid, mucus, uterine lavage fluid, a vaginal swab, a nasal swab, an oral swab, a tissue scraping, hair, a finger print, urine, stool, vitreous humor, peritoneal wash, sputum, bronchial lavage, oral lavage, pleural lavage, gastric lavage, gastric juice, bile, pancreatic duct lavage, bile duct lavage, common bile duct lavage, gall bladder fluid, synovial fluid, an infected wound, a non-infected wound, an archeological sample, a forensic sample, a water sample, a tissue sample, a food sample, a bioreactor sample, a plant sample, a fingernail scraping, semen, prostatic fluid, fallopian tube lavage, a cell free nucleic acid, a nucleic acid within a cell, a metagenomics sample, a lavage of an implanted foreign body, a nasal lavage, intestinal fluid, epithelial brushing, epithelial lavage, tissue biopsy, an autopsy sample, a necropsy sample, an organ sample, a human identification ample, an artificially produced nucleic acid sample, a synthetic gene sample, a nucleic acid data storage sample, tumor tissue, and any combination thereof. In other embodiments, a sample is or comprises at least one of a microorganism, a plant-based organism, or any collected environmental sample (e.g., water, soil, archaeological, etc.).

Modifications

In accordance with various embodiments, nucleic acid material may receive one or more modifications prior to, substantially simultaneously, or subsequent to, any particular step, depending upon the application for which a particular provided method or composition is used.

In some embodiments, a modification may be or comprise repair of at least a portion of the nucleic acid material. While any application-appropriate manner of nucleic acid repair is contemplated as compatible with some embodiments, certain exemplary methods and compositions therefore are described below and in the Examples.

By way of non-limiting example, in some embodiments, DNA repair enzymes, such as Uracil-DNA Glycosylase (UDG), Formamidopyrimidine DNA glycosylase (FPG), and 8-oxoguanine DNA glycosylase (OGG1), can be utilized to correct DNA damage (e.g., in vitro DNA damage). In some embodiments, these DNA repair enzymes, for example, are glycoslyases that remove damaged bases from DNA. For example, UDG removes uracil that results from cytosine deamination (caused by spontaneous hydrolysis of cytosine) and FPG removes 8-oxo-guanine (e.g., most common DNA lesion that results from reactive oxygen species). FPG also has lyase activity that can generate 1 base gap at abasic sites. Such abasic sites will subsequently fail to amplify by PCR, for example, because the polymerase fails copy the template. Accordingly, the use of such DNA damage repair enzymes can effectively remove damaged DNA that doesn't have a true mutation, but might otherwise be undetected as an error following sequencing and duplex sequence analysis.

In further embodiments, sequencing reads generated from the processing steps discussed herein can be further filtered to eliminate false mutations by trimming ends of the reads most prone to artifacts. For example, DNA fragmentation can generate single-strand portions at the terminal ends of double-stranded molecules. These single-stranded portions can be filled in (e.g., by Klenow) during end repair. In some instances, polymerases make copy mistakes in these end-repaired regions leading to the generation of “pseudoduplex molecules.” These artifacts can appear to be true mutations once sequenced. These errors, as a result of end repair mechanisms, can be eliminated from analysis post-sequencing by trimming the ends of the sequencing reads to exclude any mutations that may have occurred, thereby reducing the number of false mutations. In some embodiments, such trimming of sequencing reads can be accomplished automatically (e.g., a normal process step). In some embodiments, a mutant frequency can be assessed for fragment end regions and if a threshold level of mutations is observed in the fragment end regions, sequencing read trimming can be performed before generating a double-strand consensus sequence read of the DNA fragments.

Some embodiments of Duplex Sequencing methods provide PCR-based targeted enrichment strategies compatible with the use of cleavable hairpin adapters for error correction. For example, sequencing enrichment strategy utilizing Separated PCRs of Linked Templates for sequencing (“SPLiT-DS”) method steps may also benefit from pre-enriched nucleic acid material using one or more of the embodiments described herein. SPLiT-DS was originally described in International Patent Publication No. WO/2018/175997, which is incorporated herein by reference in its entirety. A SPLiT-DS approach can begin with labelling (e.g., tagging) fragmented double-stranded nucleic acid material (e.g., from a DNA sample) with molecular barcodes in a similar manner as described above and with respect to a standard Duplex Sequencing library construction protocol. In some embodiments, the double-stranded nucleic acid material may be fragmented (e.g., such as with cell free DNA, damaged DNA, etc.); however, in other embodiments, various steps can include fragmentation of the nucleic acid material using mechanical shearing such as sonication, or other DNA cutting methods, such as described further herein. Aspects of labelling the fragmented double-stranded nucleic acid material can include end-repair and 3′-dA-tailing, if required in a particular application, followed by ligation of the double-stranded nucleic acid fragments with Duplex Sequencing adapters (e.g., cleavable hairpin adapters, Y-shaped adapters, etc.). In other embodiments, an endogenous or a combination of exogenous and endogenous SMI sequence for uniquely relating information from both strands of an original nucleic acid molecule can also be used in combination with physical linkage of the first and second strands. Following ligation of adapter molecules to the double-stranded nucleic acid material, the method can continue with amplification (e.g., PCR amplification, rolling circle amplification, multiple displacement amplification, isothermal amplification, bridge amplification, surface-bound amplification, etc.).

Kits with Reagents

Aspects of the present technology further encompass kits for conducting various aspects of Duplex Sequencing methods (also referred to herein as a “DS kit”). In some embodiments, a kit may comprise various reagents along with instructions for conducting one or more of the methods or method steps disclosed herein for nucleic acid extraction, nucleic acid library preparation, amplification (e.g. PCR, bridge amplification), cleavage of linked nucleic acid complexes, and sequencing. In one embodiment, a kit may further include a computer program product (e.g., coded algorithm to run on a computer, an access code to a cloud-based server for running one or more algorithms, etc.) for analyzing sequencing data (e.g., raw sequencing data, sequencing reads, etc.) to determine, for example, a variant allele, mutation, etc., associated with a sample and in accordance with aspects of the present technology. Kits may include DNA standards and other forms of positive and negative controls.

In some embodiments, a DS kit may comprise reagents or combinations of reagents suitable for performing various aspects of sample preparation (e.g., tissue manipulation, DNA extraction, DNA fragmentation), nucleic acid library preparation, amplification, cleavage and on-sequencer surface processing steps and sequencing (e.g., enzymes, dNTPs, wash buffers, etc.). For example, a DS kit may optionally comprise one or more DNA extraction reagents (e.g., buffers, columns, etc.) and/or tissue extraction reagents. Optionally, a DS kit may further comprise one or more reagents or tools for fragmenting double-stranded DNA, such as by physical means (e.g., tubes for facilitating acoustic shearing or sonication, nebulizer unit, etc.) or enzymatic means (e.g., enzymes for random or semi-random genomic shearing and appropriate reaction enzymes). For example, a kit may include DNA fragmentation reagents for enzymatically fragmenting double-stranded DNA that includes one or more of enzymes for targeted digestion (e.g., restriction endonucleases, CRISPR/Cas endonuclease(s) and RNA guides, and/or other endonucleases), double-stranded Fragmentase cocktails, single-stranded DNase enzymes (e.g., mung bean nuclease, Si nuclease) for rendering fragments of DNA predominantly double-stranded and/or destroying single-stranded DNA, and appropriate buffers and solutions to facilitate such enzymatic reactions.

In an embodiment, a DS kit comprises primers and adapters for preparing a nucleic acid sequence library from a sample that is suitable for performing Duplex Sequencing process steps to generate error-corrected (e.g., high accuracy) sequences of double-stranded nucleic acid molecules in the sample. For example, the kit may comprise at least one pool of adapter molecules comprising a linker domain (e.g., hairpin adapter), at least one pool of adapter molecules comprising a double-stranded portion and a single-stranded portion (e.g., “Y” shape adapter) or the tools (e.g., single-stranded oligonucleotides) for the user to create it. In some embodiments, the pool of adapter molecules will comprise single molecule identifier (SMI) sequences or a suitable number of substantially unique SMI sequences such that a plurality of nucleic acid molecules in a sample can be substantially uniquely labeled following attachment of the adapter molecules, either alone or in combination with unique features of the fragments to which they are ligated. One experienced in the art of molecular tagging will recognize that what entails a “suitable” number of SMI sequences will vary by multiple orders of magnitude depending on various specific factors (input DNA, type of DNA fragmentation, average size of fragments, complexity vs repetitiveness of sequences being sequenced within a genome etc.) Optionally, the adaptor molecules further include one or more PCR primer binding sites, one or more sequencing primer binding sites, or both. In another embodiment, a DS kit does not include adapter molecules comprising SMI sequences or barcodes, but instead includes conventional adapter molecules (e.g., Y-shape sequencing adapters, etc.) and various method steps can utilize endogenous SMIs and/or physical location on a sequencing surface to relate molecule sequence reads. In some embodiments, the adapter molecules are indexing adapters and/or comprise an indexing sequence. In other embodiments, indexes are added to specific samples through “tailing in” by PCR using primers supplied in a kit

In an embodiment, a DS kit comprises a set of adapter molecules each having a non-complementary region and/or some other strand defining element (SDE), or the tools for the user to create it (e.g., single-stranded oligonucleotides). In another embodiment, the kit comprises at least one set of adapter molecules wherein at least a subset of the adapter molecules each comprise at least one SMI and at least one SDE, or the tools to create them. In some embodiments, the subsets of adapter molecules may be configured with ligateable ends (e.g., blunt ends, overhangs, substantially or partially unique sticky ends, etc.) Additional features for primers and adapters for preparing a nucleic acid sequencing library from a sample that is suitable for performing Duplex Sequencing process steps are described above as well as disclosed in U.S. Pat. No. 9,752,188, International Patent Publication No. WO2017/100441, and International Patent Application No. PCT/US18/59908 (filed Nov. 8, 2018), all of which are incorporated by reference herein in their entireties.

In an embodiment, a DS kit comprises reagents for processing steps occurring on a sequencing surface, such as cleavage facilitators (e.g., enzymes, non-enzymatic solutions, light, hybridizing oligonucleotides, etc.) and anti-cleavage facilitators (e.g., enzymes including catalytically inactive enzymes, hybridizing oligonucleotides, and the like), as well as other wash solutions for performing various steps of the methods.

Additionally, a kit may further include DNA quantification materials such as, for example, DNA binding dye such as SYBR™ green or SYBR™ gold (available from Thermo Fisher Scientific, Waltham, Mass.) or the alike for use with a Qubit™ fluorometer (e.g., available from Thermo Fisher Scientific, Waltham, Mass.), or PicoGreen™ dye (e.g., available from Thermo Fisher Scientific, Waltham, Mass.) for use on a suitable fluorescence spectrometer or a real-time PCR machine or digital-droplet PCR machine. Other reagents suitable for DNA quantification on other platforms are also contemplated. Further embodiments include kits comprising one or more of nucleic acid size selection reagents (e.g., Solid Phase Reversible Immobilization (SPRI) magnetic beads, gels, columns), columns for target DNA capture using bait/pray hybridization, qPCR reagents (e.g., for copy number determination) and/or digital droplet PCR reagents. In some embodiments, a kit may optionally include one or more of library preparation enzymes (ligase, polymerase(s), endonuclease(s), reverse transcriptase for e.g., RNA interrogations), dNTPs, buffers, capture reagents (e.g., beads, surfaces, coated tubes, columns, etc.), indexing primers, amplification primers (PCR primers) and sequencing primers. In some embodiments, a kit may include reagents for assessing types of DNA damage such as an error-prone DNA polymerase and/or a high-fidelity DNA polymerase. Additional additives and reagents are contemplated for PCR or ligation reactions in specific conditions (e.g., high GC rich genome/target).

In an embodiment, the kits further comprise reagents, such as DNA error correcting enzymes that repair DNA sequence errors that interfere with polymerase chain reaction (PCR) processes (versus repairing mutations leading to disease). By way of non-limiting example, the enzymes comprise one or more of the following: monofunctional uracil-DNA glycosylase (hSMUG1), Uracil-DNA Glycosylase (UDG), N-glycosylase/AP-lyase NEIL 1 protein (hNEIL1), Formamidopyrimidine DNA glycosylase (FPG), 8-oxoguanine DNA glycosylase (OGG1), human apurinic/apyrimidinic endonuclease (APE 1), endonuclease III (Endo III), endonuclease IV (Endo IV), endonuclease V (Endo V), endonuclease VIII (Endo VIII), T7 endonuclease I (T7 Endo I), T4 pyrimidine dimer glycosylase (T4 PDG), human single-strand-selective human alkyladenine DNA glycosylase (hAAG), etc., among other glycosylases, lyases, endonucleases and exonucleases etc.; and can be utilized to correct DNA damage (e.g., in vitro or in vivo DNA damage). Some of such DNA repair enzymes, for example, are glycoslyases that remove damaged bases from DNA. For example, UDG removes uracil that results from cytosine deamination (caused by spontaneous hydrolysis of cytosine) and FPG removes 8-oxo-guanine (e.g., most common DNA lesion that results from reactive oxygen species). FPG also has lyase activity that can generate 1 base gap at abasic sites. Such abasic sites will subsequently fail to amplify by PCR, for example, because the polymerase fails copy the template. Accordingly, the use of such DNA damage repair enzymes, and/or others listed here and as known in the art, can effectively remove damaged DNA that does not have a true mutation but might otherwise be undetected as an error.

The kits may further comprise appropriate controls, such as DNA amplification controls, nucleic acid (template) quantification controls, sequencing controls, nucleic acid molecules derived from a similar biological source (e.g., a healthy subject). In some embodiments, a kit may include a control population of cells. Accordingly, a kit could include suitable reagents (test compounds, nucleic acid, control sequencing library, etc.) for providing controls that would yield expected Duplex Sequencing results that would determine protocol authenticity for samples comprising a rare genetic variant (e.g., nucleic acid molecules comprising disease-associated variants/mutations that can be spiked into or included in the sample preparation steps). In some embodiments, a kit may include reference sequence information. In some embodiments, a kit may include sequence information useful for identifying one or more DNA variants in a population of cells or in a cell-free DNA sample. In an embodiment, the kit comprises containers for shipping samples, storage material for stabilizing samples, material for freezing samples, such as cell samples, for analysis to detect DNA variants in a subject sample. In another embodiment, a kit may include nucleic acid contamination control standards (e.g., hybridization capture probes with affinity to genomic regions in an organism that is different than the test or subject organism).

The kit may further comprise one or more other containers comprising materials desirable from a commercial and user standpoint, including PCR and sequencing buffers, diluents, subject sample extraction tools (e.g. syringes, swabs, etc.), and package inserts with instructions for use. In addition, a label can be provided on the container with directions for use, such as those described above; and/or the directions and/or other information can also be included on an insert which is included with the kit; and/or via a website address provided therein. The kit may also comprise laboratory tools such as, for example, sample tubes, plate sealers, microcentrifuge tube openers, labels, magnetic particle separator, foam inserts, ice packs, dry ice packs, insulation, etc.

The kits may further include pre-packaged or application-specific functionalized surfaces for use in amplification of the sequencing library. In one embodiment, the functionalized surface may include a surface suitable for performing sequencing reactions therein. The functionalized surface may be pre-configured with bound oligonucleotides suitable for bridge amplification of the sequencing library (e.g., the surface comprises a distributed lawn of bound oligonucleotides complementary to sequence domains in one or more of the adapter sets). In one embodiment, the functionalized surface is a flow cell configured for use in a sequencing system as described below.

The kits may further comprise a computer program product installable on an electronic computing device (e.g. laptop/desktop computer, tablet, etc.) or accessible via a network (e.g. remote server, cloud computing), wherein the computing device or remote server comprises one or more processors configured to execute instructions to perform operations comprising Duplex Sequencing analysis steps. For example, the processors may be configured to execute instructions for processing raw or unanalyzed sequencing reads to generate Duplex Sequencing data. In additional embodiments, the computer program product may include a database comprising subject or sample records (e.g., information regarding a particular subject or sample or groups of samples) and empirically-derived information regarding targeted regions of DNA. The computer program product is embodied in a non-transitory computer readable medium that, when executed on a computer, performs steps of the methods disclosed herein.

The kits may further comprise include instructions and/or access codes/passwords and the like for accessing remote server(s) (including cloud-based servers) for uploading and downloading data (e.g., sequencing data, reports, other data) or software to be installed on a local device. All computational work may reside on the remote server and be accessed by a user/kit user via internet connection, etc.

The kits may be suitable for use with sequencing systems optimized for use with the methods and reagents described herein. For example, the sequencing systems and associated sequencing reagents may be configured to perform step-wise sequencing reactions that provide for intervening processing steps. In one embodiment, the sequencing system may provide delivery systems for cleavage facilitator delivery, anti-cleavage facilitatory delivery, enzyme solution delivery, oligonucleotide delivery, wash buffers, and the like. Likewise, the sequencing system may include appropriate controls (e.g., manual, automatic, semi-automatic, etc.) and internal programing for processing step time, temperature, pH, concentration and the like.

### EXAMPLES

In addition to the various aspects, embodiments, examples, etc. described herein, the present disclosure includes the following exemplary aspects (“E”) numbered E1 through E87. This list of aspects is presented as an exemplary list and the application is not limited to these aspects.

E1. A Method of sequencing a double-stranded target nucleic acid molecule, the method comprising:
(a) amplifying a physically-linked nucleic acid complex on a surface to produce physically-linked nucleic acid complex amplicons bound to the surface in both a forward orientation and a reverse orientation, wherein the physically-linked nucleic acid complex comprises (i) the double-stranded target nucleic acid molecule, (ii) a first adapter comprising a linker domain on a first end of the double-stranded target nucleic acid molecule, and (iii) a second adapter having a double-stranded portion and a single-stranded portion on a second end of the double-stranded target nucleic acid molecule;
(b) removing either (i) the physically-linked nucleic acid complex amplicons bound to the surface in the reverse orientation or (ii) the physically-linked nucleic acid complex amplicons bound to the surface in the forward orientation;
(c) cleaving a portion of the remaining bound physically-linked nucleic acid complex amplicons to provide a subset of single-stranded amplicons comprising information from one strand and a subset of physically-linked nucleic acid complex amplicons;
(d) sequencing the subset of single-stranded amplicons to provide a sequencing read derived from an original strand of the double-stranded target nucleic acid molecule;
(e) amplifying the subset of physically-linked nucleic acid complex amplicons on the surface;
(f) removing the physically-linked nucleic acid complex amplicons that are in the other orientation;
(g) cleaving the remaining bound physically-linked nucleic acid complex amplicons to provide single-stranded amplicons comprising information from the other strand; and
(h) sequencing the single-stranded amplicons to provide sequencing reads derived from the other original strand of the double-stranded target nucleic acid molecule.

E2. A method of sequencing a double-stranded target nucleic acid molecule, the method comprising
(a) amplifying a physically-linked nucleic acid complex on a surface to produce a cluster of physically-linked nucleic acid complex amplicons bound to the surface, wherein the physically-linked nucleic acid complex comprises (i) the double-stranded target nucleic acid molecule, (ii) a first adapter comprising a linker domain on one end of the double-stranded target nucleic acid molecule, and (iii) a second adapter having a double-stranded portion and a single-stranded portion on the other end of the double-stranded target nucleic acid molecule;
(b) removing either the physically-linked nucleic acid complex amplicons bound to the surface at (i) a 5′ end of the physically-linked nucleic acid complex amplicons or (ii) a 3′ end of the physically-linked nucleic acid complex amplicons;
(c) cleaving at least a portion of the remaining bound physically-linked nucleic acid complex amplicons at a cleavage site to provide single-stranded amplicons comprising sequence information derived from one original strand of the double-stranded target nucleic acid molecule; and
(d) sequencing the single-stranded amplicons to provide a sequencing read derived from the one original strand of the double-stranded target nucleic acid molecule.

E3. The method of E2, wherein cleaving at least a portion of the remaining bound physically-linked nucleic acid complex amplicons comprises preserving at least one physically-linked nucleic acid complex amplicon bound to the surface.

E4. The method of E3, further comprising:
(e) amplifying the at least one physically-linked nucleic acid complex amplicon on the surface to repopulate the cluster of physically-linked nucleic acid complex amplicons bound to the surface;
(f) removing the physically-linked nucleic acid complex amplicons that are in the other orientation not removed in (b);
(g) cleaving the remaining bound physically-linked nucleic acid complex amplicons to provide single-stranded amplicons comprising information derived from the other original strand of the double-stranded target nucleic acid molecule; and
(h) sequencing the single-stranded amplicons to provide a sequencing read derived from the other original strand of the double-stranded target nucleic acid molecule.

E5. The method of any of the proceeding examples, further comprising comparing the sequence read from the one original strand to the sequence read from the other original strand to generate a consensus sequence for the double-stranded target nucleic acid molecule.

E6. The method of any of E1-E4, further comprising:
identifying sequence variations in the sequence read from the one original strand and the sequence read from the other original strand, wherein the sequence variations from the one original strand and the other original strand are consistent sequence variations; or
eliminating or discounting sequence variations that occur in the one original strand and not the other original strand.

E7. The method of any of E1-E4, further comprising:
comparing the sequence read from the one original strand to the sequence read from the other original strand;
identifying a nucleotide position that does not agree between the sequence read from the one original strand to the sequence read from the other original strand; and
generating an error-corrected sequence of the double-stranded target nucleic acid molecule by discounting. eliminating, or correcting the nucleotide position identified that does not agree.

E8. A method of sequencing a population of double-stranded target nucleic acid molecules, each comprising a first strand and a second strand, the method comprising:
(a) amplifying a plurality of physically-linked nucleic acid complexes on a surface to produce a plurality of clonal clusters, each clonal cluster comprising a plurality of physically-linked nucleic acid complex amplicons each comprising a first strand amplicon and a second strand amplicon, wherein each physically-linked nucleic acid complex comprises (i) a double-stranded target nucleic acid molecule from the population, (ii) a first adapter comprising a linker domain attached to a first end of the double-stranded target nucleic acid molecule, and (iii) a second adapter having a double-stranded portion and a single-stranded portion attached to a second end of the double-stranded target nucleic acid molecule;
(b) removing either the physically-linked nucleic acid complex amplicons from each clonal cluster bound to the surface in the (i) reverse orientation or (ii) in the forward orientation;
(c) cleaving a portion of the remaining surface bound physically-linked nucleic acid complex amplicons remaining after (b) and thereby physically separating the first strand amplicons and the second strand amplicons;
(d) removing the unbound physically separated first or second strand amplicons; and
(e) sequencing the remaining physically separated first or second strand amplicons bound to the surface to produce a nucleic acid sequence read of the first strand or the second strand for each clonal cluster on the surface.

E9. The method of E8, wherein cleaving at least a portion of the remaining bound physically-linked nucleic acid complex amplicons comprises preserving at least one physically-linked nucleic acid complex amplicon in at least some of the clonal clusters bound to the surface.

E10. The method of E9, further comprising:
(f) in at least some of the clonal clusters, amplifying the at least one physically-linked nucleic acid complex amplicon on the surface to repopulate the clonal clusters of physically-linked nucleic acid complex amplicons bound to the surface;
(g) removing the physically-linked nucleic acid complex amplicons that are in the other orientation from step (b);
(h) removing the unbound physically separated first or second strand amplicons;
(i) cleaving the remaining bound physically-linked nucleic acid complex amplicons remaining after (h) and thereby physically separating the first strand amplicons and the second strand amplicons; and
(j) sequencing the remaining physically separated first or second strand amplicons bound to the surface to produce a nucleic acid sequence read of the first strand or the second strand for each clonal cluster on the surface.

E11. A method of sequencing a population of double-stranded target nucleic acid molecules, each comprising a first strand and a second strand, the method comprising:
(a) amplifying a plurality of physically-linked nucleic acid complexes bound on a surface to produce a plurality of clusters, each cluster comprising a plurality of physically-linked nucleic acid complex amplicons representing an original double-stranded target nucleic acid molecule, wherein each physically-linked nucleic acid complex amplicon comprises a first strand amplicon and a second strand amplicon, and wherein each physically-linked nucleic acid complex comprises a double-stranded target nucleic acid molecule from the population attached to (i) a first adapter comprising a linker domain between the first strand and the second strand at one end and (ii) a second adapter having a double-stranded portion and a single-stranded portion at the other end;
(b) cleaving the surface bound physically-linked nucleic acid complex amplicons and thereby physically separating the first strand amplicons and the second strand amplicons;
(c) removing the unbound physically separated first strand amplicons and/or the unbound physically separated second strand amplicons, wherein the remaining amplicons bound to the surface comprise (i) the physically separated first strand amplicons and (ii) the physically separated second strand amplicons;
(d) sequencing the physically separated first strand amplicons bound to the surface to produce a nucleic acid sequence read of the first strand for each cluster on the surface; and
(e) sequencing the physically separated second strand amplicons bound to the surface to produce a nucleic acid sequence read of the second strand for each cluster on the surface.

E12. The method of E10 or E11, further comprising: for at least some of the clusters on the surface, comparing the nucleic acid sequence read of the first strand to the nucleic acid sequence read of the second strand to generate an error-corrected sequence read of an original double-stranded target nucleic acid molecule.

E13. The method of any one of E10-E12, further comprising relating the nucleic acid sequence read of the first strand of an original double-stranded target nucleic acid molecule from the population to the nucleic acid sequence read of the second strand of the same original double-stranded target nucleic acid molecule using a unique molecular identifier (UMI).

E14. The method of E13, wherein the UMI comprises a physical location on the surface.

E15. The method of E14, wherein the UMI comprises a tag sequence, a molecule-specific feature, cluster location on the surface or a combination thereof.

E16. The method of E15, wherein the molecule-specific feature comprises nucleic acid mapping information against a reference sequence, sequence information at or near the ends of the double-stranded target nucleic acid molecule, a length of the double-stranded target nucleic acid molecule, or a combination thereof.

E17. The method of any one of E10-E16, further comprising differentiating the nucleic acid sequence read of the first strand of an original double-stranded target nucleic acid molecule from the nucleic acid sequence read of the second strand from the same original double-stranded target nucleic acid molecule using a strand defining element (SDE).

E18. The method of E17, wherein the SDE is the association of sequence read information with step (e) and step (j) of E10, or with step (d) and (e) of E11.

E19. The method of E17, wherein the SDE comprises a portion of an adapter sequence.

E20. The method of any one of E8-E19, wherein sequencing the physically separated first strand amplicons or the second strand amplicons comprises sequencing by synthesis.

E21. The method of any one of E8-E20, further comprising:
preparing the physically-linked nucleic acid complexes by ligating the first adapter and the second adapter to each of a plurality of double-stranded target nucleic acid molecules in the population; and
presenting the physically-linked nucleic acid complexes to the surface, the surface having a plurality of bound oligonucleotides at least partially complimentary to the single-stranded portion of the second adapters such that a plurality of physically-linked nucleic acid complexes are captured on the surface via hybridization to the plurality of bound oligonucleotides.

E22. The method of E21, further comprising amplifying the physically-linked nucleic acid complexes prior to the presenting step.

E23. The method of E22, wherein amplifying the physically-linked nucleic acid complexes prior to the presenting step comprises PCR amplification or circle amplification.

E24. The method of any one of E21-E23, wherein the physically-linked nucleic acid complexes are captured in both a forward and a reverse orientation on the surface.

E25. The method of any one of E8-E24, wherein the amplification step in (a) comprises bridge amplification.

E26. The method of any one of E8-E25, further comprising:
for at least some of the double-stranded target nucleic acid molecules in the population-(i) comparing the sequence read from the first strand to the sequence read from the second strand;
(ii) identifying a nucleotide position that does not agree between the sequence read from the first strand and the sequence read from the second strand; and
(iii) generating an error-corrected sequence read of the double-stranded target nucleic acid molecule by discounting, eliminating, or correcting the identified nucleotide position that does not agree.

E27. The method of any one of E1-E26, wherein the first adapter comprises a cleavable site or motif.

E28. The method of any of E1-E27, wherein the first adapter and the second adapter each comprise a sequencing primer binding site and optionally, a single molecule identifier (SMI) sequence.

E29. The method of any one of E1-E27, wherein the second adapter comprises a sequencing primer binding site, an amplification primer binding site, an indexing sequence or any combination thereof.

E30. The method of any one of E1-E29, wherein the linker domain comprises a cleavage site.

E31. The method of any one of E1-E29, wherein the first adapter comprises a cleavable domain.

E32. The method of any one of E1-E31, wherein the first adapter comprises a hairpin loop structure comprising a self-complementary stem portion and a single-stranded nucleotide loop portion.

E33. The method of E32, wherein the single-stranded nucleotide loop portion comprises a cleavable domain.

E34. The method of E32, wherein the stem portion comprises a cleavable domain.

E35. The method of E33 or E34, wherein the cleavable domain comprises an enzyme recognition site.

E36. The method of E35, wherein the enzyme recognition site is an endonuclease recognition site.

E37. The method of E36, wherein the endonuclease is a restriction enzyme or a targeted endonuclease.

E38. The method of any one of E1-E37, wherein the second adapter is a “Y” shaped adapter.

E39. The method of E38, wherein one or both arms of the Y-shaped adapter can hybridize to oligonucleotides bound to the surface.

E40. The method of any of E1-E39, wherein the single-stranded portion of the second adapter comprises a first arm having a first primer binding site and a second arm having a second primer binding site.

E41. The method of E40, wherein, when denatured, the physically-linked double-stranded nucleic acid complex comprises from 5′ to 3′ or from 3′ to 5′: the first primer binding site, the first strand, the first adapter comprising the linker domain, the second strand, and the second primer binding site.

E42. The method of any of E1-E41, wherein the surface is a sequencing surface.

E43. The method of any of E1-E42, wherein the surface is a flow cell.

E44. The method of any of E1-E43, wherein the surface is a surface of a bead.

E45. The method of any of E1-E44, wherein the amplification is selected from the group consisting of PCR amplification, isothermal amplification, polony amplification, cluster amplification, and bridge amplification.

E46. The method of any of E1-E45, wherein the amplification is bridge amplification on the surface.

E47. The method of any of E8-E46, wherein one or more of the plurality of first strand amplicons and/or the plurality of second strand amplicons is bound to the surface in a forward orientation.

E48. The method of any of E8-E46, wherein one or more of the plurality of first strand amplicons and/or the plurality of second strand amplicons is bound to the surface in a reverse orientation.

E49. The method of any of E8-E48, further comprising flowing the plurality of physically-linked double stranded nucleic acid complexes over the surface prior to the amplification in (a).

E50. The method of any of E1-E49, wherein the surface comprises a plurality of one or more bound oligonucleotides at least partially complimentary to one or more regions of the second adapter.

E51. The method of E50, wherein the plurality of one or more bound oligonucleotides is at least partially complimentary to the single-stranded portion of the second adapter.

E52. The method of any of E1-E51, wherein a first strand and a second strand of the physically-linked nucleic acid complex are amplified via multiple amplification reactions in step (a) to generate a cluster of the physically-linked nucleic acid complex amplicons on the surface.

E53. The method of any of E8-E52, wherein the first strand and the second strand of each of the plurality of physically-linked nucleic acid complexes are amplified in step (a) to generate the plurality of clusters on the surface simultaneously.

E54. The method of any of E1-E8 and E12-E53, wherein cleaving a portion of the bound physically-linked nucleic acid complex amplicons comprises inefficiently cleaving at a cleavable site in the first adapter resulting in both cleaved nucleic acid complexes and uncleaved nucleic acid complexes within each cluster on the surface.

E55. The method of E54, wherein the ratio of uncleaved nucleic acid complexes of all nucleic acid complexes within each cluster on the flow cell is 1%, 5%, 10%, 20%, 30%, 40%, 45%, or 50%.

E56. The method of E54 or E55, wherein the cleaved nucleic acid complexes are cleaved at a cleavable site in the linker domain of the first adapter by a cleavage facilitator.

E57. The method of E56, wherein the cleavage is a site-directed enzymatic reaction.

E58. The method of E56 or E57, wherein the cleavage facilitator is an endonuclease.

E59. The method of E58, wherein the endonuclease is a restriction site endonuclease or a targeted endonuclease.

E60. The method of E56 or E57, wherein the cleavage facilitator is selected from the group consisting of a ribonucleoprotein, a Cas enzyme, a Cas9-like enzyme, a meganuclease, a transcription activator-like effector-based nuclease (TALEN), a zinc-finger nuclease, an argonaute nuclease or a combination thereof.

E61. The method of E56 or E57, wherein the cleavage facilitator comprises a CRISPR-associated enzyme.

E62. The method of E56 or E57, wherein the cleavage facilitator comprises Cas9 or CPF1 or a derivative thereof.

E63. The method of E56 or E57, wherein the cleavage facilitator comprises a nickase or nickase variant.

E64. The method of E56, wherein the cleavage facilitator comprises a chemical process.

E65. The method of any of E54-E64, wherein the amount of uncleaved nucleic acid complexes remaining on the surface can be scaled by controlling the amount or concentration of the cleavage facilitator being introduced for site-directed cleavage or by controlling the amount of time the cleavage facilitator is being introduced for site-directed cleavage.

E66. The method of any of E54-E63, wherein the uncleaved nucleic acid complexes are protected by addition of an anti-cleavage facilitator before or during the cleavage step.

E67. The method of E66, wherein the anti-cleavage facilitator comprises an anti-cleavage motif in the linker domain of the first adapter.

E68. The method of E67, wherein the cleavable site is already present in the linker domain of the first adapter and the anti-cleavage motif is created by hybridization of an oligonucleotide comprising an at least partially complementary sequence to the linker domain of the first adapter.

E69. The method of E66-E68, wherein cleaving a portion of the bound physically-linked nucleic acid complex amplicons further comprises:
(i) introducing the anti-cleavage facilitator; and
(ii) either following or simultaneously with (i), introducing the cleavage facilitator,
wherein interaction with the anti-cleavage facilitator protects a physically-linked nucleic acid complex amplicon from cleavage.

E70. The method of E54-E63, wherein the cleavable site is created by hybridization of an oligonucleotide comprising an at least partially complementary sequence to the linker domain of the first adapter and wherein physically-linked nucleic acid complex amplicons not hybridized with the oligonucleotide, are not cleaved.

E71. The method of E54-E63, wherein the cleavable site is created by hybridization of a first oligonucleotide comprising an at least partially complementary sequence to the linker domain of the adapter and an anti-cleavage motif is created by hybridization of a second oligonucleotide comprising an at least partially complementary sequence to the linker domain of the adapter, and wherein cleaving a portion of the bound physically-linked nucleic acid complex amplicons further comprises:
(i) introducing a mixture of the first and second oligonucleotides; and
(ii) introducing the cleavage facilitator.

E72. The method of E71, wherein either the first oligonucleotide or the second oligonucleotide is methylated.

E73. The method of E70 or E71, wherein the hybridization can be scaled by controlling the amount or concentration of the oligonucleotides being introduced for hybridization or by controlling the amount of time the oligonucleotides are being introduced for hybridization.

E74. The method of any of E67, E68 or E71-E73, wherein the anti-cleavage motif comprises an oligonucleotide sequence having a bulky adduct or a side chain that prevents access to the cleavage site.

E75. The method of any of E67, E68 or E71-E73, wherein the anti-cleavage motif comprises an oligonucleotide sequence having one or more mismatches that prevent the cleavage facilitator from recognizing the cleavage site.

E76. The method of any of E67, E68 or E71-E73, wherein the anti-cleavage motif comprises one or more of the following: an oligonucleotide sequence having a nucleoside analogue, an abasic site, a nucleotide analogue, and a peptide-nucleic acid bond.

E77. The method of E54-E63, wherein the cleaved nucleic acid complexes are cleaved at a cleavable site in the first adapter by a catalytically active enzyme and the uncleaved nucleic acid complexes are protected from cleavage in the first adapter by a catalytically inactive enzyme.

E78. The method of any of E54-E63, wherein the cleavage site is in a self-complementary portion of the first adapter or a single-stranded portion of the first adapter.

E79. The method of E78 wherein the cleavage site is available when the physically-linked nucleic acid complex amplicons are in a self-hybridized configuration on the surface.

E80. The method of any of E54-E63, wherein the cleavage site is available when the physically-linked nucleic acid complex amplicons are in a double-stranded bridge amplified configuration.

E81. The method of any of E8-E80, further comprising selectively enriching for physically-linked nucleic acid complexes having one or more targeted genomic regions prior to step (a) to provide a plurality of enriched physically-linked nucleic acid complexes.

E82. A kit able to be used in error corrected duplex sequencing of double-stranded nucleic acid molecules, the kit comprising:
at least one set of sequencing primers;
a set of first adapter molecules comprising a linker domain;
a set of second adapter molecules comprising a double stranded portion and a single stranded portion configured to be immobilized on a surface for amplification;
wherein the primers and adaptor molecules are able to be used in error corrected duplex sequencing experiments; and instructions on methods of use of the kit in conducting error corrected duplex sequencing of nucleic acid extracted from a biological sample.

E83. The kit of E82, further comprising a cleavage facilitator.

E84. The kit of E82 or E83, wherein the linker domain has a cleavable motif.

E85. The kit of any one of E82-E84, further comprising a anti-cleavage facilitator.

E86. The kit of any one of E82-E85, further comprising a computer program product embodied in a non-transitory computer readable medium that, when executed on a computer or remote computing server, performs steps of determining an error-corrected duplex sequencing read for one or more double-stranded nucleic acid molecules in a sample.

E87. A sequencing system, comprising:
a sequencing surface comprising covalently bound oligonucleotides;
a delivery system for delivering sequencing reagents to the sequencing surface;
a delivery system for delivering a cleavage facilitator to the sequencing surface; and
a computing network for transmitting information relating to sequencing data, wherein the information includes one or more of raw sequencing data, duplex sequencing data, and sample information.

### Conclusion

The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments. All references cited herein are incorporated by reference as if fully set forth herein.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. A method of sequencing a double-stranded target nucleic acid molecule, the method comprising:
(a) amplifying a physically-linked nucleic acid complex on a surface to produce physically-linked nucleic acid complex amplicons bound to the surface in both a forward orientation and a reverse orientation, wherein the physically-linked nucleic acid complex comprises (i) the double-stranded target nucleic acid molecule, (ii) a first adapter comprising a linker domain on a first end of the double-stranded target nucleic acid molecule, and (iii) a second adapter having a double-stranded portion and a single-stranded portion on a second end of the double-stranded target nucleic acid molecule;
(b) removing either (i) the physically-linked nucleic acid complex amplicons bound to the surface in the reverse orientation or (ii) the physically-linked nucleic acid complex amplicons bound to the surface in the forward orientation;
(c) cleaving a portion of the remaining bound physically-linked nucleic acid complex amplicons to provide a subset of single-stranded amplicons comprising information from one strand and a subset of physically-linked nucleic acid complex amplicons;
(d) sequencing the subset of single-stranded amplicons to provide a sequencing read derived from an original strand of the double-stranded target nucleic acid molecule;
(e) amplifying the subset of physically-linked nucleic acid complex amplicons on the surface;
(f) removing the physically-linked nucleic acid complex amplicons that are in the other orientation;
(g) cleaving the remaining bound physically-linked nucleic acid complex amplicons to provide single-stranded amplicons comprising information from the other strand; and
(h) sequencing the single-stranded amplicons to provide sequencing reads derived from the other original strand of the double-stranded target nucleic acid molecule.

2. A method of sequencing a double-stranded target nucleic acid molecule, the method comprising:
(a) amplifying a physically-linked nucleic acid complex on a surface to produce a cluster of physically-linked nucleic acid complex amplicons bound to the surface, wherein the physically-linked nucleic acid complex comprises (i) the double-stranded target nucleic acid molecule, (ii) a first adapter comprising a linker domain on one end of the double-stranded target nucleic acid molecule, and (iii) a second adapter having a double-stranded portion and a single-stranded portion on the other end of the double-stranded target nucleic acid molecule;
(b) removing either the physically-linked nucleic acid complex amplicons bound to the surface at (i) a 5′ end of the physically-linked nucleic acid complex amplicons or (ii) a 3′ end of the physically-linked nucleic acid complex amplicons;
(c) cleaving at least a portion of the remaining bound physically-linked nucleic acid complex amplicons at a cleavage site to provide single-stranded amplicons comprising sequence information derived from one original strand of the double-stranded target nucleic acid molecule; and
(d) sequencing the single-stranded amplicons to provide a sequencing read derived from the one original strand of the double-stranded target nucleic acid molecule.

3. The method of claim 2, wherein cleaving at least a portion of the remaining bound physically-linked nucleic acid complex amplicons comprises preserving at least one physically-linked nucleic acid complex amplicon bound to the surface.

4. The method of claim 3, further comprising:
(e) amplifying the at least one physically-linked nucleic acid complex amplicon on the surface to repopulate the cluster of physically-linked nucleic acid complex amplicons bound to the surface;
(f) removing the physically-linked nucleic acid complex amplicons that are in the other orientation not removed in (b);
(g) cleaving the remaining bound physically-linked nucleic acid complex amplicons to provide single-stranded amplicons comprising information derived from the other original strand of the double-stranded target nucleic acid molecule; and
(h) sequencing the single-stranded amplicons to provide a sequencing read derived from the other original strand of the double-stranded target nucleic acid molecule.

5. The method of any of the proceeding claims, further comprising comparing the sequence read from the one original strand to the sequence read from the other original strand to generate a consensus sequence for the double-stranded target nucleic acid molecule.

6. The method of any of claims 1-4, further comprising:
identifying sequence variations in the sequence read from the one original strand and the sequence read from the other original strand, wherein the sequence variations from the one original strand and the other original strand are consistent sequence variations; or
eliminating or discounting sequence variations that occur in the one original strand and not the other original strand.

7. The method of any of claims 1-4, further comprising:
comparing the sequence read from the one original strand to the sequence read from the other original strand;
identifying a nucleotide position that does not agree between the sequence read from the one original strand to the sequence read from the other original strand; and
generating an error-corrected sequence of the double-stranded target nucleic acid molecule by discounting. eliminating, or correcting the nucleotide position identified that does not agree.

8. A method of sequencing a population of double-stranded target nucleic acid molecules, each comprising a first strand and a second strand, the method comprising:
(a) amplifying a plurality of physically-linked nucleic acid complexes on a surface to produce a plurality of clonal clusters, each clonal cluster comprising a plurality of physically-linked nucleic acid complex amplicons each comprising a first strand amplicon and a second strand amplicon, wherein each physically-linked nucleic acid complex comprises (i) a double-stranded target nucleic acid molecule from the population, (ii) a first adapter comprising a linker domain attached to a first end of the double-stranded target nucleic acid molecule, and (iii) a second adapter having a double-stranded portion and a single-stranded portion attached to a second end of the double-stranded target nucleic acid molecule;
(b) removing either the physically-linked nucleic acid complex amplicons from each clonal cluster bound to the surface in the (i) reverse orientation or (ii) in the forward orientation;
(c) cleaving a portion of the remaining surface bound physically-linked nucleic acid complex amplicons remaining after (b) and thereby physically separating the first strand amplicons and the second strand amplicons;
(d) removing the unbound physically separated first or second strand amplicons; and
(e) sequencing the remaining physically separated first or second strand amplicons bound to the surface to produce a nucleic acid sequence read of the first strand or the second strand for each clonal cluster on the surface.

9. The method of claim 8, wherein cleaving at least a portion of the remaining bound physically-linked nucleic acid complex amplicons comprises preserving at least one physically-linked nucleic acid complex amplicon in at least some of the clonal clusters bound to the surface.

10. The method of claim 9, further comprising:
(f) in at least some of the clonal clusters, amplifying the at least one physically-linked nucleic acid complex amplicon on the surface to repopulate the clonal clusters of physically-linked nucleic acid complex amplicons bound to the surface;
(g) removing the physically-linked nucleic acid complex amplicons that are in the other orientation from step (b);
(h) removing the unbound physically separated first or second strand amplicons;
(i) cleaving the remaining bound physically-linked nucleic acid complex amplicons remaining after (h) and thereby physically separating the first strand amplicons and the second strand amplicons; and
(j) sequencing the remaining physically separated first or second strand amplicons bound to the surface to produce a nucleic acid sequence read of the first strand or the second strand for each clonal cluster on the surface.

11. A method of sequencing a population of double-stranded target nucleic acid molecules, each comprising a first strand and a second strand, the method comprising:
(a) amplifying a plurality of physically-linked nucleic acid complexes bound on a surface to produce a plurality of clusters, each cluster comprising a plurality of physically-linked nucleic acid complex amplicons representing an original double-stranded target nucleic acid molecule, wherein each physically-linked nucleic acid complex amplicon comprises a first strand amplicon and a second strand amplicon, and wherein each physically-linked nucleic acid complex comprises a double-stranded target nucleic acid molecule from the population attached to (i) a first adapter comprising a linker domain between the first strand and the second strand at one end and (ii) a second adapter having a double-stranded portion and a single-stranded portion at the other end;
(b) cleaving the surface bound physically-linked nucleic acid complex amplicons and thereby physically separating the first strand amplicons and the second strand amplicons;
(c) removing the unbound physically separated first strand amplicons and/or the unbound physically separated second strand amplicons, wherein the remaining amplicons bound to the surface comprise (i) the physically separated first strand amplicons and (ii) the physically separated second strand amplicons;
(d) sequencing the physically separated first strand amplicons bound to the surface to produce a nucleic acid sequence read of the first strand for each cluster on the surface; and
(e) sequencing the physically separated second strand amplicons bound to the surface to produce a nucleic acid sequence read of the second strand for each cluster on the surface.

12. The method of claim 10 or claim 11, further comprising: for at least some of the clusters on the surface, comparing the nucleic acid sequence read of the first strand to the nucleic acid sequence read of the second strand to generate an error-corrected sequence read of an original double-stranded target nucleic acid molecule.

13. The method of any one of claims 10-12, further comprising relating the nucleic acid sequence read of the first strand of an original double-stranded target nucleic acid molecule from the population to the nucleic acid sequence read of the second strand of the same original double-stranded target nucleic acid molecule using a unique molecular identifier (UMI).

14. The method of claim 13, wherein the UMI comprises a physical location on the surface.

15. The method of claim 14, wherein the UMI comprises a tag sequence, a molecule-specific feature, cluster location on the surface or a combination thereof.

16. The method of claim 15, wherein the molecule-specific feature comprises nucleic acid mapping information against a reference sequence, sequence information at or near the ends of the double-stranded target nucleic acid molecule, a length of the double-stranded target nucleic acid molecule, or a combination thereof.

17. The method of any one of claims 10-16, further comprising differentiating the nucleic acid sequence read of the first strand of an original double-stranded target nucleic acid molecule from the nucleic acid sequence read of the second strand from the same original double-stranded target nucleic acid molecule using a strand defining element (SDE).

18. The method of claim 17, wherein the SDE is the association of sequence read information with step (e) and step (j) of claim 10, or with step (d) and (e) of claim 11.

19. The method of claim 17, wherein the SDE comprises a portion of an adapter sequence.

20. The method of any one of claims 8-19, wherein sequencing the physically separated first strand amplicons or the second strand amplicons comprises sequencing by synthesis.

21. The method of any one of claims 8-20, further comprising:
preparing the physically-linked nucleic acid complexes by ligating the first adapter and the second adapter to each of a plurality of double-stranded target nucleic acid molecules in the population; and
presenting the physically-linked nucleic acid complexes to the surface, the surface having a plurality of bound oligonucleotides at least partially complimentary to the single-stranded portion of the second adapters such that a plurality of physically-linked nucleic acid complexes are captured on the surface via hybridization to the plurality of bound oligonucleotides.

22. The method of any one of claims 8-21, wherein the amplification step in (a) comprises bridge amplification.

23. The method of any one of claims 8-22, further comprising:
for at least some of the double-stranded target nucleic acid molecules in the population
(i) comparing the sequence read from the first strand to the sequence read from the second strand;
(ii) identifying a nucleotide position that does not agree between the sequence read from the first strand and the sequence read from the second strand; and
(iii) generating an error-corrected sequence read of the double-stranded target nucleic acid molecule by discounting, eliminating, or correcting the identified nucleotide position that does not agree.

24. The method of any one of claims 1-23, wherein the first adapter comprises a cleavable site or motif.

25. The method of any one of claims 1-24, wherein the first adapter comprises a cleavable domain.

26. The method of any one of claims 1-25, wherein the first adapter comprises a hairpin loop structure comprising a self-complementary stem portion and a single-stranded nucleotide loop portion.

27. The method of claim 26, wherein the cleavable domain is in the single-stranded nucleotide loop portion or the stem portion.

28. The method of claim 33, wherein the cleavable domain comprises an enzyme recognition site.

29. The method of claim 28, wherein the enzyme recognition site is targeted by a restriction enzyme or a targeted endonuclease.

30. The method of any of claims 1-29, wherein the single-stranded portion of the second adapter comprises a first arm having a first primer binding site and a second arm having a second primer binding site.

31. The method of claim 30, wherein, when denatured, the physically-linked double-stranded nucleic acid complex comprises from 5′ to 3′ or from 3′ to 5′: the first primer binding site, the first strand, the first adapter comprising the linker domain, the second strand, and the second primer binding site.

32. The method of any of the previous claims, wherein the surface is a sequencing surface.

33. The method of any of one of claims 8-32, further comprising flowing the plurality of physically-linked double stranded nucleic acid complexes over the surface prior to the amplification in (a).

34. The method of any of the previous claims, wherein the surface comprises a plurality of one or more bound oligonucleotides at least partially complimentary to one or more regions of the second adapter.

35. The method of claim 34, wherein the plurality of one or more bound oligonucleotides is at least partially complimentary to the single-stranded portion of the second adapter.

36. The method of any one of claims 1-35, wherein a first strand and a second strand of the physically-linked nucleic acid complex are amplified via multiple amplification reactions in step (a) to generate a cluster of the physically-linked nucleic acid complex amplicons on the surface.

37. The method of any of claim 8-36, wherein the first strand and the second strand of each of the plurality of physically-linked nucleic acid complexes are amplified in step (a) to generate the plurality of clusters on the surface simultaneously.

38. The method of any one of claims 1-8 and 12-37, wherein cleaving a portion of the bound physically-linked nucleic acid complex amplicons comprises inefficiently cleaving at a cleavable site in the first adapter resulting in both cleaved nucleic acid complexes and uncleaved nucleic acid complexes within each cluster on the surface.

39. The method of claim 38, wherein the ratio of uncleaved nucleic acid complexes of all nucleic acid complexes within each cluster on the flow cell is 1%, 5%, 10%, 20%, 30%, 40%, 45%, or 50%.

40. The method of claim 38 or 39, wherein the cleaved nucleic acid complexes are cleaved at a cleavable site in the linker domain of the first adapter by a cleavage facilitator.

41. The method of claim 40, wherein the cleavage is a site-directed enzymatic reaction.

42. The method of claim 40 or claim 41, wherein the cleavage facilitator is an endonuclease.

43. The method of claim 40 or claim 41, wherein the cleavage facilitator comprises a CRISPR-associated enzyme.

44. The method of claim 40 or claim 41, wherein the cleavage facilitator comprises a nickase or nickase variant.

45. The method of claim 40, wherein the cleavage facilitator comprises a chemical process.

46. The method of any one of claims 38-45, wherein the amount of uncleaved nucleic acid complexes remaining on the surface can be scaled by controlling the amount or concentration of the cleavage facilitator being introduced for site-directed cleavage or by controlling the amount of time the cleavage facilitator is being introduced for site-directed cleavage.

47. The method of any one of claims 38-45, wherein the uncleaved nucleic acid complexes are protected by addition of an anti-cleavage facilitator before or during the cleavage step.

48. The method of claim 47, wherein cleaving a portion of the bound physically-linked nucleic acid complex amplicons further comprises:
(i) introducing the anti-cleavage facilitator; and
(ii) either following or simultaneously with (i), introducing the cleavage facilitator,
wherein interaction with the anti-cleavage facilitator protects a physically-linked nucleic acid complex amplicon from cleavage.

49. The method of claim 38-44, wherein the cleavable site is created by hybridization of an oligonucleotide comprising an at least partially complementary sequence to the linker domain of the first adapter and wherein physically-linked nucleic acid complex amplicons not hybridized with the oligonucleotide, are not cleaved.

50. The method of claim 38-44, wherein the cleavable site is created by hybridization of a first oligonucleotide comprising an at least partially complementary sequence to the linker domain of the adapter and an anti-cleavage motif is created by hybridization of a second oligonucleotide comprising an at least partially complementary sequence to the linker domain of the adapter, and wherein cleaving a portion of the bound physically-linked nucleic acid complex amplicons further comprises:
(i) introducing a mixture of the first and second oligonucleotides; and
(ii) introducing the cleavage facilitator.

51. The method of claims 38-44, wherein the cleaved nucleic acid complexes are cleaved at a cleavable site in the first adapter by a catalytically active enzyme and the uncleaved nucleic acid complexes are protected from cleavage in the first adapter by a catalytically inactive enzyme.

52. The method of any one of claims 38-44, wherein the cleavage site is in a self-complementary portion of the first adapter or a single-stranded portion of the first adapter.

53. The method of claim 52 wherein the cleavage site is available when the physically-linked nucleic acid complex amplicons are in a self-hybridized configuration on the surface.

54. The method of any one of claims 38-44, wherein the cleavage site is available when the physically-linked nucleic acid complex amplicons are in a double-stranded bridge amplified configuration.
